(19) United States
(12) Patent Application Publication
(10) Pub. No.: US 2013/0323279 A1
(43) Pub. Date: Dec. 5, 2013

(54) Human Endogenous Retrovirus Polypeptide Compositions and Methods of Use Thereof

(71) Applicants: The J. David Gladstone Institutes, San Francisco, CA (US); Albert Einstein College of Medicine of Yeshiva University, Bronx, NY (US); The Regents of the University of California, Oakland, CA (US); The Governing Council of University of Toronto, Toronto (CA)

(72) Inventors: Douglas F. Nixon, San Francisco, CA (US); Keith E. Garrison, San Francisco, CA (US); Duncan A. Meiklejohn, San Francisco, CA (US); Mario A. Ostrowski, Toronto (CA); R. Bradley Jones, Toronto (CA); Ashish Agrawal, San Francisco, CA (US); Jack Lenz, Bronx, NY (US); Seth Rakoff-Nahoum, New Haven, CT (US); Frederick M. Hecht, San Francisco, CA (US)

(73) Assignees: The J. David Gladstone Institutes, San Francisco, CA (US); Albert Einstein College of Medicine of Yeshiva University, Bronx, NY (US); The Regents of the University of California, Oakland, CA (US); The Governing Counicl of University of Toronto, Toronto (CA)

(21) Appl. No.: 13/891,970

(22) Filed: May 10, 2013

Related U.S. Application Data
(62) Division of application No. 11/880,126, filed on Jul. 19, 2007, now abandoned.
(60) Provisional application No. 60/832,465, filed on Jul. 21, 2006.

(51) Int. Cl. A61K 39/21

(52) U.S. Cl. CPC A61K 39/21
USPC 424/207.1

(57) ABSTRACT

The present invention provides isolated HERV polypeptides; and compositions, including immunogenic compositions, comprising a HERV polypeptide. The present invention provides immunogenic compositions comprising a nucleic acid comprising a nucleotide sequence encoding a HERV polypeptide. The immunogenic compositions are useful for stimulating a T cell immune response to a lentiviral peptide. The present invention further provides methods of stimulating an immune response in an individual to a retrovirus- or lentivirus-infected cell. The present invention further provides methods of treating cancers in which HERV polypeptides are expressed. Also provided are methods of treating disorders, involving decreasing an immune response to a HERV polypeptide.

### CROSS-REFERENCE

This application claims the benefit of U.S. Provisional Patent Application No. 60/832,465, filed Jul. 21, 2006, which application is incorporated herein by reference in its entirety.

### BACKGROUND

Human endogenous retrovirus sequences make up 8.29% of the draft human genome. Their prevalence has resulted from the accumulation of past retroviral infectious agents that have entered the germline and established a truce with the host cell. Genes co-opted by the host from endogenous retroviruses are found to be active participants in some cellular processes including viral defense by Fv1 and Fv4 in the mouse, and cellular fusion in human placental development mediated through syncitin. Although-HERV transcripts have been detected in both normal and cancerous tissues, including T cells, their role in normal cell function and carcinogenesis is unclear. While the cellular conditions that promote HERV transcription are not well understood, the APOBECs have been shown to play a role in the control of endogenous retroviruses.

### LITERATURE

Griffiths (2001) Genome Biology 2:1017.1-1017.5; Müller and De Boer (2006) PLoS Pathogens 2:0149; Nelson et al. (2003) J. Clin. Pathol: Mol. Pathol. 56:11-18; Contreras-Galindo et al. (2007) AIDS Res. Human Retrovir. 23:116-122; U.S. Patent Publication No. 2005/0118573; Rakoff-Nahoum et al. (2006) AIDS Res. Human Retrovir. 22:52-56; Schiavetti et al. (2002) Cancer Res. 62:5510-5516; Büscher et al. (2005) Cancer Res. 65:4172; Clerici et al. (1999) J. Neuroimmunol. 99:173.

### SUMMARY OF THE INVENTION

The present invention provides isolated HERV polypeptides; and compositions, including immunogenic compositions, comprising a HERV polypeptide. The present invention provides immunogenic compositions comprising a nucleic acid comprising a nucleotide sequence encoding a HERV polypeptide. The immunogenic compositions are useful for stimulating a T cell immune response to a lentiviral peptide. The present invention further provides methods of stimulating an immune response in an individual to a retrovirus- or lentivirus-infected cell. The present invention further provides methods of treating cancers in which HERV polypeptides are expressed. Also provided are methods of treating disorders, involving decreasing an immune response to a HERV polypeptide.

### BRIEF DESCRIPTION OF THE DRAWINGS

FIGS. 1A and 1B depict expression of HERV-K transcripts in HIV positive and negative individuals' plasma.

FIG. 2 depicts HERV/HIV amino acid alignments of HIV HXB-2 and various HERV insertions showing segments of the Gag and Reverse Transcriptase proteins.

FIG. 3 depicts ELISPOT T cell responses to HERV and HIV antigens in HIV positive and negative individuals.

FIG. 4 depicts an inverse correlation between anti-HERV T cell responses and HIV-1 plasma viral load.

FIG. 5 depicts the results of a ⁵¹Cr release assay to measure cytotoxicity of HERV-L IQ10-specific CD8⁺ T cells.

FIG. 6 depicts an amino acid sequence of HERV-K reverse transcriptase.

FIG. 7A depicts an amino acid sequence of a HERV-L reverse transcriptase.

FIG. 7B depicts a nucleotide sequence encoding a HERV-L reverse transcriptase.

FIG. 8A depicts an amino acid sequence of a HERV-H envelope.

FIG. 8B depicts a nucleotide sequence encoding a HERV-L envelope.

### DEFINITIONS

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as CD4⁺ T lymphocytes, CD8⁺ T lymphocytes, glial cells, macrophages, tumor cells, peripheral blood mononuclear cells (PBMC), and the like. The term “biological sample” encompasses a clinical sample, and also includes cells in culture, cell supernatants, tissue samples, organs, bone marrow, blood, plasma, serum, cerebrospinal fluid, and the like.

The term “retrovirus” is well known in the art, and includes single-stranded, positive sense, enveloped RNA viruses that include, e.g., the genus Gammaretrovirus (e.g., murine mammary tumor virus); the genus Epsilonretrovirus; the genus Alpharetrovirus (e.g., avian leukosis virus); the genus Betaretrovirus; the genus Deltaretrovirus (e.g., bovine leukemia virus; human T-lymphotrophic virus (HTLV)); the genus Lentivirus; and the genus Spumavirus. The term “lentivirus,” as used herein, refers to a genus of viruses of the Retroviridae family, and includes human immunodeficiency virus-1 (HIV-1); human immunodeficiency virus-2 (HIV-2); simian immunodeficiency virus. (SIV); and feline immunodeficiency virus (FIV).

“Gene delivery vehicle” refers to a construct which is capable of delivering, and, within some embodiments expressing, one or more gene(s) or nucleotide sequence(s) of interest in a host cell. Representative examples of such vehicles include viral vectors, nucleic acid expression vectors, naked DNA, and certain eukaryotic cells (e.g., producer cells).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct-the expression thereof. Thus, for example, intervening untranslated yet transcribed sequences can be present between a promoter sequence and the coding sequence and the promoter sequence can still be considered “operably linked” to the coding sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells. An isolated polypeptide will in some embodiments be synthetic. “Synthetic polypeptides” are assembled from amino acids, and are chemically synthesized in vitro, e.g., cell-free chemical synthesis, using procedures known to those skilled in the art.

By “purified” is meant a compound of interest (e.g., a polypeptide) has been separated from components that accompany it in nature. “Purified” can also be used to refer to a compound of interest separated from components that can accompany it during manufacture (e.g., in chemical synthesis). In some embodiments, a compound is substantially pure when it is at least 50% to 60%, by weight, free from organic molecules with which it is naturally associated or with which it is associated during manufacture. In some embodiments, the preparation is at least 75%, at least 90%, at least 95%, or at least 99%, by weight, of the compound of interest. A substantially pure compound can be obtained, for example, by extraction from a natural source (e.g., bacteria), by chemically synthesizing a compound, or by a combination of purification and chemical modification. A substantially pure compound can also be obtained by, for example, enriching a sample having a compound that binds an antibody of interest. Purity can be measured by any appropriate method, e.g., chromatography, mass spectroscopy, high performance liquid chromatography analysis, etc.

The term “heterologous,” as used herein in the context of a HERV polypeptide, where a HERV polypeptide fusion protein comprises a HERV polypeptide and a heterologous polypeptide, refers to a polypeptide that is other than a HERV polypeptide, e.g., a polypeptide that is not normally associated with a HERV polypeptide. For example, a heterologous polypeptide bears no significant amino acid sequence identity to the HERV antigenic polypeptide, e.g., the heterologous polypeptide has less than about 50%, less than about 40%, less than about 30%, or less than about 20% amino acid sequence identity to the HERV antigenic polypeptide.

An “antigen” is defined herein to include any substance that may be specifically bound by an antibody molecule. An “immunogen” is an antigen that is capable of initiating lymphocyte activation resulting in an antigen-specific immune response.

By “epitope” is meant a site on an antigen to which specific B cells and/or T cells respond. The term is also used interchangeably with “antigenic determinant” or “antigenic determinant site.” B cell epitope sites on proteins, polysaccharides, or other biopolymers may be composed of moieties from different parts of the macromolecule that have been brought together by folding. Epitopes of this kind are referred to as conformational or discontinuous epitopes, since the site is composed of segments of the polymer that are discontinuous in the linear sequence but are continuous in the folded conformation(s). Epitopes that are composed of single segments of biopolymers or other molecules are termed continuous or linear epitopes. T cell epitopes are generally linear peptides. Antibodies that recognize the same epitope can be identified in a simple immunoassay showing the ability of one antibody to block the binding of another antibody to a target antigen.

The terms “cancer,” “neoplasm,” and “tumor” are used interchangeably herein to refer to cells which exhibit relatively autonomous growth, so that they exhibit an aberrant growth phenotype characterized by a significant loss of control of cell proliferation. Cells of interest for treatment in the present application include precancerous, malignant, pre-metastatic, metastatic, and non-metastatic cells, as well as carcinoma in situ.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

The terms “subject,” “individual,” “host,” and “patient” are used interchangeably herein to refer to a mammal, including, but not limited to, murines (rats, mice), felines, non-human primates (e.g., simians), humans, canines, ungulates, etc.

The terms “treatment,” “treating,” “treat,” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a human endogenous retrovirus polypeptide” includes a plurality of such polypeptides and reference to “the immunogenic composition” includes reference to one or more immunogenic compositions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides isolated HERV polypeptides; and compositions, including immunogenic compositions, comprising a HERV polypeptide. The present invention provides immunogenic compositions comprising a nucleic acid comprising a nucleotide sequence encoding a HERV polypeptide. The immunogenic compositions are useful for stimulating a T cell immune response to a lentiviral peptide. The present invention further provides methods of stimulating an immune response in an individual to a retrovirus- or lentivirus-infected cell. The present invention further provides methods of treating cancers in which HERV polypeptides are expressed by cancerous cells. Also provided are methods of treating disorders, involving decreasing an immune response to a HERV polypeptide.

In some embodiments, a subject immunogenic composition induces a T cell immune response specific for a lentivirus-infected cell, e.g., a human immunodeficiency virus (HIV)-infected cell. Epitopes displayed by a HERV polypeptide stimulate or enhance a T cell immune response to the epitopes. Where the HERV epitopes are also present on the surface of a lentivirus-infected cell, a T cell response to the lentivirus-infected cell also occurs. A “T cell immune response” includes one or more of 1) an increase in the number and/or activity of CD4⁺ T cells specific for the HERV epitope; 2) an increase in the number and/or activity (e.g., cytotoxicity) of CD8⁺ T cells specific for the HERV epitope; and 3) secretion of cytokines that induce or are indicative of a Th2-type immune response. Cytokines that induce or are indicative of a Th2 immune response include, but are not limited to, interferon-gamma (IFN-γ), IL-2, and tumor necrosis factor-alpha (TNF-α). T cell immune responses that are stimulated with a subject immunogenic composition include a mucosal T cell immune response and a systemic T cell immune response.

A subject immunogenic composition may be formulated in any of a variety of ways, including a formulation suitable for intravenous administration, subcutaneous administration, or other parenteral route of administration; a formulation suitable for administration to a mucosal tissue; and the like. The present invention provides pharmaceutical formulations comprising a subject immunogenic composition.

The present invention further provides HERV polypeptide compositions that are suitable for use in monitoring a patient's response to treatment for a lentivirus infection (e.g., an HIV infection). Thus, the present invention further provides methods for monitoring a patient's response to treatment for a lentivirus infection (e.g., an HIV infection).

### Isolated HERV Polypeptides

The present invention provides isolated HERV polypeptides, and compositions comprising the HERV polypeptides. Isolated HERV polypeptides find use in, e.g., generating immunogenic compositions (e.g., for enhancing an immune response in an individual to a HERV polypeptide); generating immunomodulatory compositions (e.g., for reducing an immune response in an individual to a HERV polypeptide; monitoring patient response to therapy, e.g., therapy for a lentivirus infection; staging a disease; detecting a disease; and for generating CD8⁺ T cells for adoptive transfer methods.

### HERV Polypeptides

HERV polypeptides include polypeptides encoded by any HERV class or group, e.g., of HERV-W, HERV-H, HERV-K, HERV-L, and HERV-S, and any subgroup thereof. HERV classes, groups, and subgroups are known in the art. See, e.g., Griffiths (2001) Genome Biology 2:1017.1-1017.5.

In some embodiments, a subject isolated HERV polypeptide comprises a polypeptide comprising from about 9, 10, 11, 12, 13-15, 15-17, 17-20, from 20 to 25, from 25 to 50, from 50 to 75, from 75 to 100, from 100 to 150, from 150 to 200, from 200 to 250, from 250 to 300, from 300 to 350, or from 350 to 400, or more, contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence of a HERV-encoded polypeptide. HERV-encoded polypeptides include a polypeptide encoded by the Gag-Pro-Pol region, and a polypeptide encoded by the env region of a HERV.

In some embodiments, a subject isolated HERV polypeptide comprises a stretch of from about 9, 10, 11, 12, 13-15, 15-17, 17-20, or from 20 to 25, or more contiguous amino acids having at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or 100% amino acid sequence identity to a stretch of the same length in an HIV-encoded protein.

A subject isolated HERV polypeptide can be from 9 amino acids in length up to the length of a naturally-occurring HERV polypeptide, e.g., a HERV polypeptide can be 9 amino acids (aa), 10 aa, 11 aa, 12-15 aa, 15-20 aa, 20-25 aa, 25-30 aa, 30-40 aa, 40-50 aa, 50-100 aa, or longer than 100 amino acids, e.g., 100 aa to 150 aa, 150 aa to 200 aa.

Exemplary, non-limiting examples of HERV-encoded polypeptides are found in GenBank Accession Nos. AAD51797 (HERV-K Gag-Pro-Pol protein); AAD51798 (HERV-K env protein); CAA13576; AJ233632; AF108843; etc.

In some embodiments, a subject isolated HERV polypeptide comprises a polypeptide comprising from about 9, 10, 11, 12, 13-15, 15-17, 17-20, from 20 to 25, from 25 to 50, from 50 to 75, from 75 to 80, or from 80 to 87 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:26:

(SEQ ID NO: 26)
IIIDLKDCFFTIPLAEQDCEKFAFTIPAINNKEPATRFQWKVLPQGMLNS
PTICQTFVGRALQPVREKFSDCYIIHCIDDILCAAET.

In some embodiments, a subject isolated HERV polypeptide comprises a polypeptide comprising from about 9, 10, 11, 12, 13-15, 15-17, 17-20, from 20 to 25, from 25 to 30, from 30 to 35, from 35 to 40, or from 40 to 43 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO: 27:

(SEQ ID NO: 27)
AAIDLANAFFSIPVHKAHKKQFAFTICVYCPASGVYQQSSFVS.

In some embodiments, a subject isolated HERV polypeptide comprises a polypeptide comprising from about 9, 10, 11, 12, 13-15, 15-17, 17-20, from 20 to 25, from 25 to 30, from 30 to 35, from 35 to 40, from 40 to 45, from 45 to 50, from 50 to 55, or from 55 to 58 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:28:

(SEQ ID NO: 28)
FAFRWQGQQYSFTVLSQGYINSPALCHNLIQRELDHFLLLQDIILVHYID
DIMLIGSS.

In some embodiments, a subject isolated HERV polypeptide comprises a polypeptide comprising from about 9, 10, 11, 12, 13-15, 15-17, 17-20, from 20 to 25, from 25 to 30, from 30 to 35, from 35 to 40, from 40 to 45, from 45 to 50, from 50 to 55, from 55 to 60, from 60 to 65, or from 65 to 71 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:29:

(SEQ ID NO: 29)
KLRLPPGYFGLLLHLSQQAMKGVTVLAGVIDLDYQDEISLLLHNRGKEEY
AWNTGDPLGCLLVLPCPVIKV.

In some embodiments, a subject isolated HERV polypeptide comprises a polypeptide comprising from about 9, 10, 11, 12, 13-15, 15-17, 17-20, from 20 to 25, from 25 to 30, or from 30 to 35 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:30:

(SEQ ID NO: 30)
YTHDRAQAVPEGTSKLHEEVAQMPMVSTPATLSLP.

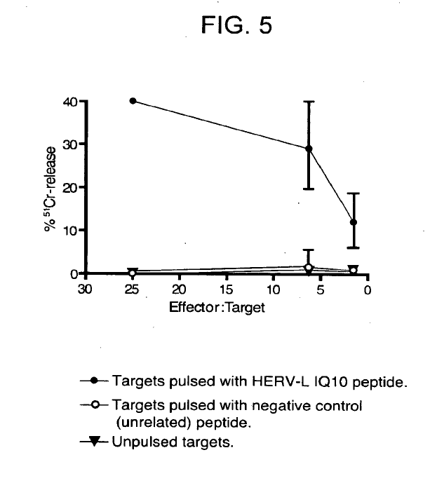

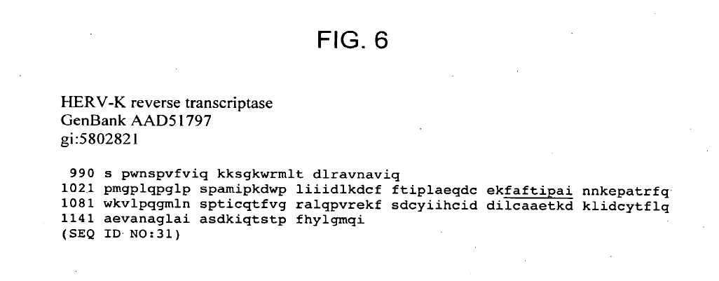

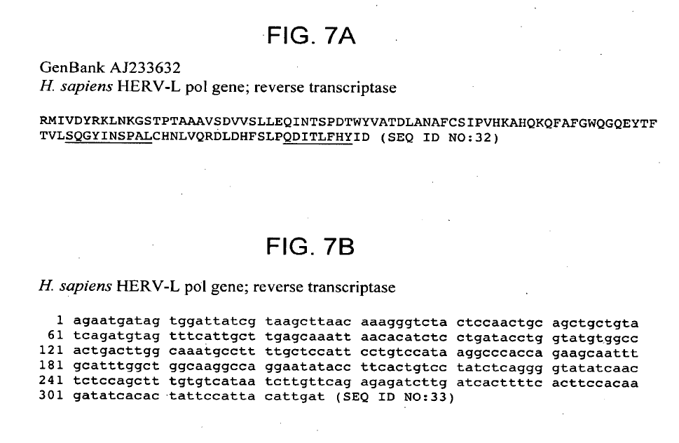

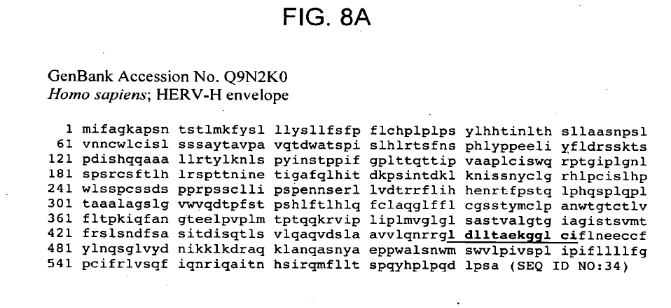

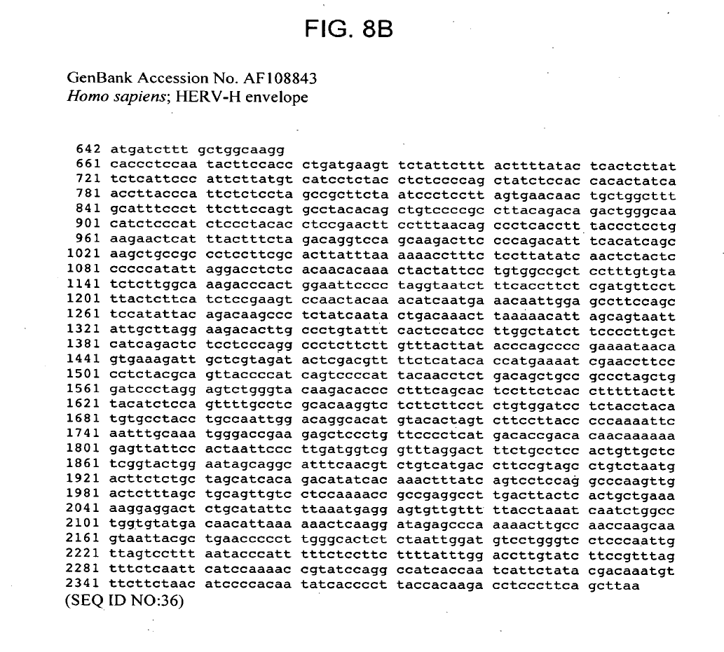

In some embodiments, a subject isolated HERV polypeptide comprises a polypeptide comprising from about 9, 10, 11, 12, 13-15, 15-17, 17-20, from 20 to 25, from 25 to 30, from 30 to 35, from 35 to 50, or from 50 to 100, or more, contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in any one of SEQ ID NOs: 31, 32, and 34, e.g., as depicted in FIGS. 6, 7A, and 8A, respectively.

In some embodiments, a subject isolated HERV polypeptide comprises one or more of the following amino acid sequences:

(SEQ ID NO: 1)
SQGYINSPAL;
(SEQ ID NO: 2)
ILVHYIDDI;
(SEQ ID NO: 3)
LQDIILVHY;
(SEQ ID NO: 4)
PMVSTPATL;
(SEQ ID NO: 5)
AAIDLANAF;
(SEQ ID NO: 6)
IPVHKAHKKQ;
(SEQ ID NO: 7)
SSGLMLMEF;
(SEQ ID NO: 8)
KIRLPPGYF;
(SEQ ID NO: 9)
DSIEGQLILK;
(SEQ ID NO: 10)
FAFTIPAI;
(SEQ ID NO: 11)
GIPYNSQGQ;
(SEQ ID NO: 12)
FEGLVDTGAD;
(SEQ ID NO: 13)
FLQFKTWWI;
(SEQ ID NO: 14)
VPLTKEQVR;
(SEQ ID NO: 15)
LDLLTAEKGGLCI;
(SEQ ID NO: 16)
TLEPIPPGE;
(SEQ ID NO: 17)
DPLAPLQLL;
(SEQ ID NO: 18)
KLLGDINWI;
(SEQ ID NO: 19)
LPHSTVKTF;
(SEQ ID NO: 20)
GPGYCSKAF;
(SEQ ID NO: 21)
IPTRHLKFY;
(SEQ ID NO: 22)
VPSFGRLSY;
(SEQ ID NO: 23)
PPTVEARYK;
(SEQ ID NO: 24)
PPESQYGYP;
and
(SEQ ID NO: 25)
YPQPPTRRL.

In certain embodiments, the following peptides are specifically excluded:

(SEQ ID NO: 13)
FLQFKTWWI;
(SEQ ID NO: 24)
PPESQYGYP;
and
(SEQ ID NO: 23)
PTVEARYK.

### Fusion Proteins

In some embodiments, a subject isolated HERV polypeptide is a fusion protein, e.g., a HERV fusion protein comprises a HERV polypeptide covalently linked to a heterologous protein, where the heterologous protein is also referred to as a “fusion partner.” In some embodiments, the fusion partner is attached to the N-terminus of the HERV protein, e.g., NH₂-fusion partner-HERV-COOH. In other embodiments, the fusion partner is attached to the C-terminus of the HERV protein, e.g., NH₂-HERV-fusion partner-COOH. In other embodiments, the fusion partner is internal to the HERV protein, e.g., NH₂-(HERV)₁-FP-(HERV₂-COOH)₂, where FP is a fusion partner, and HERV₁ and HERV₂ are N-terminal and C-terminal regions, respectively, of HERV.

Suitable fusion partners include, but are not limited to, immunological tags such as epitope tags, including, but not limited to, hemagglutinin, FLAG, myc, and the like; proteins that provide for a detectable signal, including, but not limited to, fluorescent proteins, enzymes (e.g., β-galactosidase, luciferase, horse radish peroxidase, alkaline phosphatase, etc.), and the like; polypeptides that facilitate purification or isolation of the fusion protein, e.g., metal ion binding polypeptides such as 6His tags, glutathione-S-transferase, and the like; polypeptides that provide for subcellular localization; and polypeptides that provide for secretion from a cell. Fusion partners that provide for a detectable signal are also referred to as “reporters.” In some embodiments, a fusion partner is an immunomodulatory polypeptide other than a HERV polypeptide, e.g., an antigen, a cytokine, etc.

### Multimerized HERV Polypeptides

In some embodiments, a subject isolated HERV polypeptide is multimerized, e.g., two or more HERV polypeptides are linked in tandem. Multimers include dimers, trimers, tetramers, pentamers, etc. Monomeric HERV polypeptides are linked to one another directly or via a linker. Thus, in some embodiments, a subject HERV polypeptide has the formula (X₁—(Y)₀₋₄₀—X₂—(Y)₀₋₄₀)ₙ, where X₁ and X₂ are HERV polypeptides, Y is a linker, and n is an integer from 1 to about 10 (e.g., n=1, 2, 3, 4, 5, 6, 7, 8, 9, or 10). Where a linker is used, Y is one or more amino acids, or other linking groups. X₁ and X₂ can be the same or different, e.g., can have the same amino acid sequence, or can differ from one another in amino acid sequence. Thus, e.g., a subject HERV polypeptide can have the formula X₁—(Y)₀₋₄₀—X₂, e.g., where the HERV polypeptide is a dimer. As another example, a subject HERV polypeptide can have the formula X₁—(Y)₀₋₄₀—X₂—(Y)₀₋₄₀-X₃, e.g., where the HERV polypeptide is a trimer.

Where Y is a spacer peptide, it is generally of a flexible nature, although other chemical linkages are not excluded. Currently, it is contemplated that the most useful linker sequences will generally be peptides of between about 2 and about 40 amino acids in length, e.g., from about 2 amino acids to about 10 amino acids, from about 10 amino acids to about 20 amino acids, or from about 6 amino acids to about 25 amino acids in length. These linkers are generally produced by using synthetic, linker-encoding oligonucleotides to couple the proteins. Peptide linkers with a degree of flexibility will generally be used. The linking peptides may have virtually any amino acid sequence, bearing in mind that the preferred linkers will have a sequence that results in a generally flexible peptide. The use of small amino acids, such as glycine and alanine, are of use in creating a flexible peptide. Exemplary peptide linkers include (Gly)₂₋₄₀, (Ser)₂₋₄₀, and (Ala)₂₋₄₀. The creation of such sequences is routine to those of skill in the art. A variety of different linkers are commercially available and are considered suitable for use according to the present invention. However, any flexible linker generally between about 2 amino acids and about 40 amino acids, e.g., from about 6 amino acids to about 10 amino acids in length may be used. Linkers may have virtually any sequence that results in a generally flexible peptide.

Linkages for homo- or hetero-polymers or for coupling to carriers can be provided in a variety of ways. For example, cysteine residues can be added at both the amino- and carboxyl-termini, where the peptides are covalently bonded via controlled oxidation of the cysteine residues. Also useful are a large number of heterobifunctional agents which generate a disulfide link at one functional group end and a peptide link at the other, including N-succidimidyl-3-(2-pyridyldithio)proprionate (SPDP). This reagent creates a disulfide linkage between itself and a cysteine residue in one protein and an amide linkage through the amino on a lysine or other free amino group in the other. A variety of such disulfide/amide forming agents are known. See, for example, Immun. Rev. 62:185 (1982). Other bifunctional coupling agents form a thioether rather than a disulfide linkage. Many of these thioether forming agents are commercially available and include reactive esters of 6-maleimidocaproic acid, 2 bromoacetic acid, 2-iodoacetic acid, 4-(N-maleimido-methyl)cyclohexane-1-carboxylic acid and the like. The carboxyl groups can be activated by combining them with succinimide or 1-hydroxy-2-nitro-4-sulfonic acid, sodium salt. A particularly preferred coupling agent is succinimidyl 4-(N-maleimidomethyl)cyclohexane-1-carboxylate (SMCC). Of course, it will be understood that linkage should not substantially interfere with either of the linked groups to function for its intended use, e.g., as an immunogen.

### Carriers

In some embodiments, a subject isolated HERV polypeptide is linked to a carrier. The term “linked,” as used herein interchangeably with the term “coupled,” refers to proximately associated, e.g., the HERV polypeptide and the carrier are in close spatial proximity. In some embodiments, the linkage is a covalent linkage. In other embodiments, the linkage is a non-covalent linkage. In some embodiments, the HERV polypeptide is linked directly to the carrier. In other embodiments, the HERV polypeptide is linked indirectly, e.g., via a linker molecule.

Examples of suitable carriers include large, slowly metabolized macromolecules such as: proteins; polysaccharides, such as sepharose, agarose, cellulose, cellulose beads and the like; polymeric amino acids such as polyglutamic acid, polylysine, and the like; amino acid copolymers; inactivated virus particles; inactivated bacterial toxins such as toxoid from diphtheria, tetanus, cholera, leukotoxin molecules; liposomes; inactivated bacteria; dendritic cells; and the like. Carriers are described in further detail below.

Suitable carriers are well known in the art, and include, e.g., thyroglobulin, albumins such as human serum albumin, tetanus toxoid; Diphtheria toxoid; polyamino acids such as poly(D-lysine:D-glutamic acid); VP6 polypeptides of rotaviruses; influenza virus hemagglutinin, influenza virus nucleoprotein; hepatitis B virus core protein, hepatitis B virus surface antigen; purified protein derivative (PPD) of tuberculin from Mycobacterium tuberculosis; inactivated Pseudomonas aeruginosa exotoxin A (toxin A); Keyhole Limpet Hemocyanin (KLH); filamentous hemagglutinin (FHA) of Bordetella pertussis; T helper cell (Th) epitopes of tetanus toxoid (TT) and Bacillus Calmette-Guerin (BCG) cell wall; recombinant 10 kDa, 19 kDa and 30-32 kDa proteins from M. leprae or from M. tuberculosis, or any combination of these proteins; and the like. See, e.g., U.S. Pat. No. 6,447,778 for a discussion of carriers methods of conjugating peptides to carriers.

Pseudomonas aeruginosa exotoxin A (toxin A) has been used effectively as a carrier in conjugate vaccines. Pseudomonas aeruginosa exotoxin A may be purified from the supernatant of fermentor-grown cultures of Pseudomonas aeruginosa PA 103. Toxin A has been classified as a superantigen based upon results in animals. Toxin A can be completely and irreversibly detoxified by covalent coupling to adipic acid dihydrazide (ADH), a 4 carbon spacer molecule. This step destroys the ADPR-transferase activity of the toxin molecule, hence rendering it nontoxic. The non-reacted hydrazide group can be used to covalently couple a polypeptide to toxin A. Toxin A may also be coupled to a polypeptide using a carbodiimide reagent.

PPD-peptide conjugates are conveniently prepared with glutaraldehyde as coupling agent. See, e.g., Rubinstein et al. (1995) AIDS 9:243-51.

The methods by which a subject polypeptide is conjugated with a carrier include disulfide linkages through a C terminal peptide cysteine linkage, coupling with glutaraldehyde solution for two hours, coupling with tyrosine, or coupling with water soluble carbodiimide.

In some embodiments, a subject isolated HERV polypeptide is lipidated. Lipidation increases a cytotoxic T cell (CTL) response to the peptide that is linked to the lipid. The lipid residue, such as palmitic acid or the like, is attached to the amino terminus of the peptide. The lipid can be attached directly to the peptide, or, indirectly via a linkage, such as a Ser-Ser, Gly, Gly-Gly, Ser linkage or the like. As another example, E. coli lipoprotein, such as tripalmitoyl-S-glycerylcysteinyl-seryl-serine (P₃ CSS), can be used to prime specific CTL when covalently attached to the peptide. See, Deres et al., Nature 342:561-564 (1989). A HERV polypeptide can be conjugated with uncharged fatty acid residues of different chain lengths and degrees, of unsaturation, ranging from acetic to stearic acid as well as to negatively charged succinyl residues via the appropriate carboxylic acid anhydrides. See, e.g., U.S. Pat. No. 6,419,931.

A subject isolated HERV polypeptide may be conjugated directly or indirectly, e.g., via a linker molecule, to a carrier. A wide variety of linker molecules are known in the art and can be used in the conjugates. The linkage from the peptide to the carrier may be through a peptide reactive side chain, or the N- or C-terminus of the peptide. A linker may be an organic, inorganic, or semi-organic molecule, and may be a polymer of an organic molecule, an inorganic molecule, or a co-polymer comprising both inorganic and organic molecules.

If present, the linker molecules are generally of sufficient length to permit the HERV polypeptide and a linked carrier to allow some flexible movement between the HERV polypeptide and the carrier. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

### Compositions

The present invention provides compositions comprising a subject isolated HERV polypeptide. Compositions comprising a HERV polypeptide can include one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; and the like. In some embodiments, as described in more detail below, a subject HERV composition is an immunogenic composition. In other embodiments, as described in more detail below, a subject HERV composition is a pharmaceutical composition, e.g., a composition comprising a HERV polypeptide and a pharmaceutically acceptable excipient.

In some embodiments, a subject composition, comprises a single type (or “species”) of HERV polypeptide, e.g., in some embodiments, the HERV polypeptides in a subject composition all comprise substantially the same amino acid sequence. In other embodiments, a subject immunogenic composition comprises two or more different HERV polypeptides, e.g., the composition comprises a population of HERV polypeptides, the member of which population can differ in amino acid sequence. A subject composition can comprise from two to about 20 different HERV polypeptides, e.g., a subject composition can comprise two, three, four, five, six, seven, eight, nine, ten, 11-15, or 15-20 different HERV polypeptides, each having an amino acid that differs from the amino acid sequences of the other HERV polypeptides. For example, in some embodiments, a subject composition comprises a first HERV polypeptide having a first amino acid sequence; and at least a second HERV polypeptide having a second amino acid sequence, where the second amino acid sequence differs from the first amino acid sequence. As another example, in some embodiments, a subject composition comprises a first HERV polypeptide having a first amino acid sequence; second HERV polypeptide having a second amino acid sequence, where the second amino acid sequence differs from the first amino acid sequence; and at least a third HERV polypeptide having a third amino acid sequence, where the third amino acid sequence differs from both the first and the second amino acid sequences. In other embodiments, a subject composition comprises a multimerized HERV polypeptide, as described above.

### Production of HERV Polypeptides

A subject HERV polypeptide can be produced in a number of ways, including, e.g., by chemical synthesis, where the HERV polypeptide is a “synthetic” polypeptide; by isolation and purification from a naturally-occurring source; and by recombinant means, where the HERV polypeptide is a “recombinant” polypeptide. Recombinant means for producing a HERV polypeptide are well known in the art, and involve genetically modifying a host cell with a polynucleotide comprising a nucleotide sequence encoding a HERV polypeptide, culturing the host cell in vitro under conditions and for a suitable time such that the HERV polypeptide is produced by the genetically modified cell, and isolating the HERV polypeptide produced by the genetically modified cell.

### Immunogenic Compositions Comprising a HERV Polypeptide

The present invention provides immunogenic compositions, comprising a HERV polypeptide, e.g., a polypeptide comprising amino acid sequences derived from or related to a human endogenous retrovirus (HERV) polypeptide. HERV polypeptides suitable for inclusion in a subject immunogenic composition are as described above.

In some embodiments, a subject immunogenic composition comprises a HERV polypeptide that comprises one or more T cell epitopes that, when presented on the surface of a lentivirus-infected cell, induce a T cell immune response specific for a lentivirus-infected cell, e.g., a human immunodeficiency virus (HIV)-infected cell. A “T cell immune response” includes one or more of: 1) an increase in the number and/or activity of CD4⁺ T cells specific for the HERV epitope; 2) an increase in the number and/or activity of CD8⁺ T cells specific for the HERV epitope; and 3) secretion of cytokines that induce or are indicative of a Th2-type immune response. Cytokines that induce or are indicative of a Th2 immune response include, but are not limited to, interferon-gamma (IFN-γ), IL-2, and tumor necrosis factor-alpha (TNF-α).

A subject immunogenic composition comprising a subject HERV polypeptide can be formulated in a number of ways, as described in more detail below. In some embodiments, a subject immunogenic composition comprises single species of HERV polypeptide, e.g., the immunogenic composition comprises a population of HERV polypeptides, substantially all of which have the same amino acid sequence. In other embodiments, a subject immunogenic composition comprises two or more different HERV polypeptides, e.g., the immunogenic composition comprises a population of HERV polypeptides, the member of which population can differ in amino acid sequence. A subject immunogenic composition can comprise from two to about 20 different HERV polypeptides, e.g., a subject immunogenic composition can comprise two, three, four, five, six, seven, eight, nine, ten, 11-15, or 15-20 different HERV polypeptides, each having an amino acid that differs from the amino acid sequences of the other HERV polypeptides. For example, in some embodiments, a subject immunogenic composition comprises a first HERV polypeptide having a first amino acid sequence; and at least a second HERV polypeptide having a second amino acid sequence, where the second amino acid sequence differs from the first amino acid sequence. As another example, in some embodiments, a subject immunogenic composition comprises a first HERV polypeptide having a first amino acid sequence; second HERV polypeptide having a second amino acid sequence, where the second amino acid sequence differs from the first amino acid sequence; and at least a third HERV polypeptide having a third amino acid sequence, where the third amino acid sequence differs from both the first and the second amino acid sequences. In other embodiments, a subject immunogenic composition comprises a multimerized HERV polypeptide, as described above.

### Adjuvants

The immunogenic compositions to be administered are provided in a pharmaceutically acceptable diluent such as an aqueous solution, e.g., a saline solution, a semi-solid form (e.g., gel), or in powder form. Such diluents can be inert, although a subject HERV composition may also include an adjuvant. Examples of known suitable adjuvants that can be used in humans include, but are not necessarily limited to, alum, aluminum phosphate, aluminum hydroxide, MF59 (4.3% w/v squalene, 0.5% w/v Tween 80, 0.5% w/v Span 85), CpG-containing nucleic acid (where the cytosine is unmethylated), QS21, MPL, 3DMPL, extracts from Aquilla, ISCOMS, LT/CT mutants, poly(D,L-lactide-co-glycolide) (PLG) microparticles, Quil A, interleukins, and the like. For non-human animals (e.g. for veterinary applications; for experimental non-human animals), one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogenic antigen.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (WO90/14837; Chapter 10 in Vaccine design: the subunit and adjuvant approach, eds. Powell & Newman, Plenum Press 1995), containing 5% Squalene, 0.5% Tween 80 (polyoxyethylene sorbitan mono-oleate), and 0.5% Span 85 (sorbitan trioleate) (optionally containing muramyl tri-peptide covalently linked to dipalmitoyl phosphatidylethanolamine (MTP-PE)) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21 or STIMULON™ (Cambridge Bioscience, Worcester, Mass.) may be used or particles generated therefrom such as ISCOMs (immunostimulating complexes), which ISCOMS may be devoid of additional detergent e.g. WO00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO00/56358; (6) combinations of 3dMPL with, for example, QS21 and/oroil-in-water emulsions e.g. EP-A-0835318, EP-A-0735898, EP-A-0761231; (7) oligonucleotides comprising CpG motifs [Krieg Vaccine 2000, 19, 618-622; Krieg Curr opin Mol Ther 2001 3:15-24; Roman et al., Nat. Med, 1997, 3, 849-854; Weiner et al., PNAS USA, 1997, 94, 10833-10837; Davis et al, J. Immunol, 1998, 160, 870-876; Chu et al., J. Exp. Med, 1997, 186, 1623-1631; Lipford et al, Ear. J. Immunol., 1997, 27, 2340-2344; Moldoveami et al., Vaccine, 1988, 16, 1216-1224, Krieg et al., Nature, 1995, 374, 546-549; Klinman et al., PNAS USA, 1996, 93, 2879-2883; Ballas et al, J. Immunol, 1996, 157, 1840-1845; Cowdery et al, J. Immunol, 1996, 156, 4570-4575; Halpern et al, Cell Immunol, 1996, 167, 72-78; Yamamoto et al, Jpn. J. Cancer Res., 1988, 79, 866-873; Stacey et al, J. Immunol., 1996, 157, 2116-2122; Messina et al, J. Immunol, 1991, 147, 1759-1764; Yi et al, J. Immunol, 1996, 157, 4918-4925; Yi et al, J. Immunol, 1996, 157, 5394-5402; Yi et al, J. Immunol, 1998, 160, 4755-4761; and Yi et al, J. Immunol, 1998, 160, 5898-5906; International patent applications WO96/02555, WO98/16247, WO98/18810, WO98/40100, WO98/55495, WO98/37919 and WO98/52581] i.e. containing at least one CG dinucleotide, where the cytosine is unmethylated; (8) a polyoxyethylene ether or a polyoxyethylene ester e.g. WO99/52549; (9) a polyoxyethylene sorbitan ester surfactant in combination with an octoxynol (WO01/21207) or a polyoxyethylene alkyl ether or ester surfactant in combination with at least one additional non-ionic surfactant such as an octoxynol (WO01/21152); (10) a saponin and an immunostimulatory oligonucleotide (e.g. a CpG oligonucleotide) (WO00/62800); (11) an immunostimulant and a particle of metal salt e.g. WO00/23105; (12) a saponin and an oil-in-water emulsion e.g. WO99/11241; (13) a saponin (e.g. QS21)+3dMPL+IM2 (optionally+a sterol) e.g. WO98/57659; (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine MTP-PE), etc.

The immunogenic compositions may be combined with a conventional pharmaceutically acceptable excipient, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antigen in these formulations can vary widely, and will be selected primarily based on fluid volumes, viscosities, body weight and the like in accordance with the particular mode of administration selected and the patient's needs. The resulting compositions may be in the form of a solution, suspension, tablet, pill, capsule, powder; gel, cream, lotion, ointment, aerosol or the like.

The protein concentration of a subject immunogenic in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

In some embodiments, a HERV polypeptide is formulated with one or more lipids. For example, liposomes of various sizes can be made. Small liposomes or vesicles formed are unilamellar and have a size in the range of about 20 to 400 nanometers and can be produced by subjecting multi-lamellar vesicles to ultrasound, by extrusion under pressure through membranes having pores of defined size, or by high pressure homogenization. Larger unilamellar liposomes having a size in the range of about 0.1 to 1 μm in diameter can be obtained when the lipid is solubilized in an organic solvent or a detergent and the solubilized agent is removed by evaporation or dialysis, respectively. The fusion of smaller unilamellar liposomes by methods requiring particular lipids or stringent dehydration-hydration conditions can yield unilamellar vessels as large or larger than cells.

Liposomes may comprise one or more cationic lipids, e.g., DDAB, dimethyldioctadecyl ammonium bromide; N-[1-(2,3-Dioloyloxy)propyl]-N,N,N-trimethylammonium methylsulfate; 1,2-diacyl-3-trimethylammonium-propanes, (including but not limited to, dioleoyl (DOTAP), dimyristoyl, dipalmitoyl, disearoyl); 1,2-diacyl-3-dimethylammonium-propanes, (including but not limited to, dioleoyl, dimyristoyl, dipalmitoyl, disearoyl) DOTMA, N-[1-[2,3-bis(oleoyloxy)]propyl]-N,N,N-trimethylammonium chloride; DOGS, dioctadecylamidoglycylspermine; DC-cholesterol, 3μ-[N—(N′,N′-dimethylaminoethane)carbamoyl]cholesterol; DOSPA, 2,3-dioleoyloxy-N-(2(sperminecarboxamido)-ethyl)-N,N-dimethyl-1-propanaminium trifluoroacetate; 1,2-diacyl-sn-glycero-3-ethylphosphocholines (including but not limited to dioleoyl (DOEPC), dilauroyl, dimyristoyl, dipalmitoyl, distearoyl, palmitoyl-oleoyl); β-alanyl cholesterol; CTAB, cetyl trimethyl ammonium bromide; diCl4-amidine, N-t-butyl-N′-tetradecyl-3-tetradecylaminopropionamidine; 14Dea2, O,O′-ditetradecanolyl-N-(trimethylammonioacetyl) diethanolamine chloride; DOSPER, 1,3-dioleoyloxy-2-(6-carboxy-spermyl)-propylamide; N,N,N′,N′-tetramethyl-N,N′-bis(2-hydroxylethyl)-2,3-dioleoyloxy-1,4-butanediammonium iodide; 1-[2-acyloxy)ethyl]2-alkyl(alkenyl)-3-(2-hydroxyethyl)imidazolinium chloride derivatives such as 1-[2-(9(Z)-octadecenoyloxy)ethyl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM); 1-[2-tetradecanoyloxy)ethyl]-2-tridecyl-3-(2-hydroxyethyl)imidazolium chloride (DMTIM)—as described in Solodin et al. (1995) Biochem. 43:13537-13544; 2,3-dialkyloxypropyl quaternary ammonium compound derivates, containing a hydroxyalkyl moiety on the quaternary amine, such as 1,2-dioleoyl-3-dimethyl-hydroxyethyl ammonium bromide (DOR1); 1,2-dioleyloxypropyl-3-dimethyl-hydroxyethyl ammonium bromide (DORIE); 1,2-dioleyloxypropyl-3-dimethyl-hydroxypropyl ammonium bromide (DOME-HP); 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxylethyl ammonium bromide (DMRIE); 1,2-dipalmityloxypropyl-3-dimethyl-hydroxyethyl ammonium bromide (DPRIE); 1,2-disteryloxypropyl-3-dimethyl-hydroxyethyl ammonium bromide (DSRIE)—as described, e.g., in Feigner et al. (1994) J. Biol. Chem. 269:2550-2561. Many of the above-mentioned lipids are available commercially from, e.g., Avanti Polar Lipids, Inc.; Sigma Chemical Co.; Molecular Probes, Inc.; Northerm Lipids, Inc.; Roche Molecular Biochemicals; and Promega Corp.

Liposomes may comprise cationic lipids alone, or in admixture with other lipids, particularly neutral lipids such as: cholesterol; 1,2-diacyl-sn-glycero-3-phosphoethanolamines, (including but not limited to dioleoyl (DOPE), 1,2-diacyl-sn-glycero-3-phosphocholines; natural egg yolk phosphatidyl choline (PC), and the like; synthetic mono- and diacyl phosphocholines (e.g., monoacyl phosphatidyl choline (MOPC)) and phosphoethanolamines. Asymmetric fatty acids, both synthetic and natural, and mixed formulations, for the above diacyl derivatives may also be included.

Other suitable liposome compositions include dimyristoylphosphatidylcholine (DMPC) and cholesterol. Such liposomes are described in, e.g., U.S. Pat. No. 5,916,588. Additional suitable liposomal compositions, and methods of preparing same, are known in the art, and are described in various publications, including, e.g., U.S. Pat. Nos. 4,241,046 and 6,355,267.

### Immunogenic Compositions Comprising HERV Polynucleotides

The present invention provides an immunogenic composition comprising a HERV polynucleotide, e.g., a polynucleotide comprising a nucleotide sequence encoding a HERV polypeptide. When administered to an individual in need thereof, the polynucleotide (the “HERV polynucleotide”) comprising a nucleotide sequence encoding a HERV polypeptide is taken up by a cell, e.g., an antigen-presenting cell, the encoded HERV polypeptide is produced in the cell, and the HERV polypeptide is processed into epitope-displaying polypeptide fragments (“epitope fragments”) that are then displayed on the surface of the cell in association with an MHC molecule. The encoded HERV polypeptide stimulates or enhances a T cell response to the epitope(s) displayed on the cell surface. Where the HERV epitopes are also present on a lentivirus-infected cell, a T cell response to the lentivirus-infected cell also occurs.

### Expression Vectors and Delivery Vehicles

In some embodiments, a HERV polynucleotide is an expression vector. The expression vector will provide a transcriptional and translational initiation region, which may be inducible or constitutive, where the coding region is operably linked under the transcriptional control of the transcriptional initiation region, and a transcriptional and translational termination region.

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Non-limiting examples of suitable eukaryotic promoters (promoters functional in a eukaryotic cell) include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A subject recombinant vector will in some embodiments include one or more selectable markers. In addition, the expression vectors will in many embodiments contain one or more selectable marker genes to provide a phenotypic trait for selection of transformed host cells such as dihydrofolate reductase or neomycin resistance for eukaryotic cell culture.

Other gene delivery vehicles and methods may be employed, including polycationic condensed DNA linked or unlinked to killed adenovirus alone, for example Curiel (1992) Hum. Gene Ther. 3:147-154; ligand linked DNA, for example see Wu (1989) J. Biol. Chem. 264:16985-16987; eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip (1994) Mol. Cell Biol. 14:2411-2418, and in Woffendin (1994) Proc. Natl. Acad. Sci. 91:1581-1585.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Nos. WO 95/13796, WO 94/23697, and WO 91/14445, and EP No. 524 968.

Liposome or lipid nucleic acid delivery vehicles can also be used. Liposome complexes for gene delivery are described in, e.g., U.S. Pat. No. 7,001,614. For example, liposomes comprising DOTAP and at least one cholesterol and/or cholesterol-derivative, present in a molar ratio range of 2.0 mM 10 mM provide an effective delivery system, e.g., where the molar ratio of DOTAP to cholesterol is 1:1 3:1. The cationic lipid N-[(2,3-dioleoyloxy)propyl]-L-lysinamide (LADOP) can be used in a composition for delivering a HERV polynucleotide, where LADOP-containing liposomes are described in, e.g., U.S. Pat. No. 7,067,697. Liposome formulations comprising amphipathic lipids having a polar headgroup and aliphatic components capable of promoting transfection are suitable for use and are described in, e.g., U.S. Pat. No. 6,433,017.

Further non-viral delivery suitable for use includes mechanical delivery systems such as the approach described in Woffendin et al. (1994) Proc. Natl. Acad. Sci. USA 91:11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT No. WO 92/11033.

### Treatment Methods

The present invention provides various treatment methods, which methods utilize a subject HERV polypeptide or a subject HERV composition. Subject treatment methods include methods of inducing an immune response in an individual to a HERV polypeptide, and methods of enhancing a subject's immune response to a HERV polypeptide, e.g., for the treatment of a retrovirus infection (e.g., a lentivirus infection), for the treatment of cancer, etc; and methods for reducing subject's immune response to a HERV polypeptide, e.g., for the treatment of an autoimmune disorder, for the treatment of schizophrenia, etc.

### Methods of Inducing or Enhancing an Immune Response to a Retrovirus-Infected Cell

The present invention provides methods for inducing, eliciting, or enhancing a T cell immune response to a retrovirus-infected cell, e.g., an HTLV-infected cell, in an individual in need thereof. The methods generally involve administering an effective amount of a subject immunogenic composition to the individual.

In some embodiments, an “effective amount” of a subject immunogenic composition is an amount that, when administered to an individual in one or more doses, reduces retroviral load in the individual by at least about 5%, at least about 10%, at least about 20%, at least about 25%, at least about 50%, at least about 75%, at least about 85%, or at least about 90%, compared to the viral load in the individual before treatment with the immunogenic composition.

In some embodiments, an “effective amount” of a subject immunogenic composition is an amount that, when administered to an individual in one or more doses, results in an increase in the number of T cells specific for a retrovirus epitope present on a retrovirus-infected cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for a retrovirus epitope present on a retrovirus-infected cell, compared with the number of T cells specific for a retrovirus epitope in the individual before treatment with the immunogenic composition.

In some embodiments, an “effective amount” of a subject immunogenic composition is an amount that, when administered to an individual in one or more doses, results in an increase in the number of CD8⁺ T cells specific for a retrovirus epitope present on a retrovirus-infected cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a retrovirus epitope present on a retrovirus-infected cell, compared with the number of CD8⁺ T cells specific for a retrovirus epitope in the individual before treatment with the immunogenic composition.

In some embodiments, e.g., where the immunogenic composition is administered to a naïve individual (i.e., an individual not infected with a retrovirus such as HTLV), an “effective amount” of a subject immunogenic composition is an amount that, when administered to an individual in one or more doses, reduces the likelihood that the individual, if later infected with a retrovirus such as HTLV, would develop disease symptoms from the retrovirus infection. In some embodiments, e.g., where the immunogenic composition is administered to a naïve individual (i.e., an individual not infected with a retrovirus), an “effective amount” of a subject immunogenic composition is an amount that, when administered to an individual in one or more doses, increases the likelihood that the individual, if later infected with a retrovirus such as HIV, would limit and/or clear the retrovirus infection.

### Methods of Inducing or Enhancing an Immune Response to a Lentivirus-Infected Cell

The present invention provides methods for inducing, eliciting, or enhancing a T cell immune response to a lentivirus-infected cell, e.g., an HIV-infected cell, in an individual in need thereof. The methods generally involve administering an effective amount of a subject immunogenic composition to the individual.

In some embodiments, an “effective amount” of a subject immunogenic composition is an amount that, when administered to an individual in one or more doses, reduces viral load in the individual by at least about 5%, at least about 10%, at least about 20%, at least about 25%, at least about 50%, at least about 75%, at least about 85%, or at least about 90%, compared to the viral load in the individual before treatment with the immunogenic composition.

In some embodiments, an “effective amount” of a subject immunogenic composition is an amount that, when administered to an individual in one or more doses, results in an increase in CD4⁺ T lymphocyte levels and function(s) in the individual. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, compared to the level of CD4⁺ T lymphocytes in the individual before treatment with the immunogenic composition. In some embodiments, an “effective amount” of a subject immunogenic composition is an amount that, when administered to an individual in one or more doses, results in a number of CD4⁺ T lymphocytes that is within the normal range, where the normal range for humans is from about 600 to about 1500 CD4⁺ T lymphocytes per mm³ blood.

In some embodiments, an “effective amount” of a subject immunogenic composition is an amount that, when administered to an individual in one or more doses, results in an increase in the number of T cells specific for a lentivirus epitope present on a lentivirus-infected cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for a lentivirus epitope present on a lentivirus-infected cell, compared with the number of T cells specific for a lentivirus epitope in the individual before treatment with the immunogenic composition.

In some embodiments, an “effective amount” of a subject immunogenic composition is an amount that, when administered to an individual in one or more doses, results in an increase in the number of CD8⁺ T cells specific for a lentivirus epitope present on a lentivirus-infected cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a lentivirus epitope present on a lentivirus-infected cell, compared with the number of CD8⁺ T cells specific for a lentivirus epitope in the individual before treatment with the immunogenic composition.

In some embodiments, e.g., where the immunogenic composition is administered to a naïve individual (i.e., an individual not infected with a lentivirus such as HIV), an “effective amount” of a subject immunogenic composition is an amount that, when administered to an individual in one or more doses, reduces the likelihood that the individual, if later infected with a lentivirus such as HIV, would develop disease symptoms from the lentivirus infection. In some embodiments, e.g., where the immunogenic composition is administered to a naïve individual (i.e., an individual not infected with a lentivirus such as HIV), an “effective amount” of a subject immunogenic composition is an amount that, when administered to an individual in one or more doses, increases the likelihood that the individual, if later infected with a lentivirus such as HIV, would limit and/or clear the lentivirus infection.

### Combination Therapies

A subject immunogenic composition can be administered in conjunction with one or more therapeutic agents for the treatment of a lentiviral infection, or for the treatment of a disorder that may accompany a lentiviral infection (e.g., a bacterial infection, a fungal infection, and the like). Therapeutic agents beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof.

### Methods of Treating Cancer

The present invention further provides methods of treating cancer in an individual, where the cancer is associated with expression of HERV. Such cancers include, but are not limited to, ovarian cancer, breast cancer, melanoma, prostate cancer, seminoma, teratoma, and testicular cancer. The methods generally involved administering to an individual in need thereof an effective amount of a subject immunogenic composition comprising one or more HERV polypeptides.

A subject method for treating cancer is useful for treating cancer that derived from a tissue comprising cells that normally express one or more HERV polypeptides. Such cancers include ovarian cancer, breast cancer, melanoma, prostate cancer, seminoma, teratoma, and testicular cancer.

In some embodiments, in the context of cancer treatment, an “effective amount” of a subject immunogenic composition is an amount that, when administered to an individual in one or more doses, reduces one or more of tumor size, cancer cell number, and cancer cell metastasis by at least about 10%, at least about 20%, at least about 30%, at least about 40%, at least about 50%, at least about 60%, at least about 70%, at least about 80%, or at least about 90%, up to total eradication of the tumor.

In some embodiments, an “effective amount” of a subject immunogenic composition is an amount that, when administered to an individual in one or more doses, results in an increase in the number of T cells specific for an epitope present on a cancer cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for an epitope present on a cancer cell, compared with the number of T cells specific for a cancer cell epitope in the individual before treatment with the immunogenic composition.

In some embodiments, an “effective amount” of a subject immunogenic composition is an amount that, when administered to an individual in one or more doses, results in an increase in the number of CD8⁺ T cells specific for an epitope present on a cancer cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a an epitope present on a cancer cell, compared with the number of CD8⁺ T cells specific for a cancer cell epitope in the individual before treatment with the immunogenic composition.

In some embodiments, a subject immunogenic composition is administered as an adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daanorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore, compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ (8) colony-stimulating factors; and (9) inhibitors of angiogenesis.

### Methods for Treating Autoimmune Disorders

The present invention provides methods of treating an autoimmune disorder in an individual, the methods generally involving administering to an individual in need thereof an amount of a subject HERV polypeptide effective to reduce a subject's immune response to a HERV polypeptide, thereby treating the autoimmune disease. Autoimmune disorders that can be treated with a subject method include, but are not limited to, multiple sclerosis, rheumatoid arthritis, systemic lupus erythematosus, and Type 1 diabetes.

In some embodiments, an effective amount of a subject HERV polypeptide is an amount that is effective to reduce a subject's immune response to a HERV polypeptide by at least about 10%, at least about 15%, at least about 20%, at least about 25%, at least about 30%, at least about 35%, at least about 40%, at least about 45%, at least about 50%, or more than 50%, compared to the level of the subject's immune response to the HERV polypeptide in absence of treatment with a subject HERV polypeptide.

In some embodiments, a subject method is effective in reducing autoreactivity, where “reducing autoreactivity” includes one or more of reducing the number of autoreactive cells; reducing the activity of an autoreactive cell; and reducing the level of autoreactive antibody. Autoreactivity depends on the interactions of a number of white blood cells, including but not limited to, T lymphocytes, B cells, natural killer (NK) cells and dendritic cells. T lymphocytes include CD4⁺ T lymphocytes and CD8⁺ lymphocytes. B cells can function both as antigen presenting cells and producers of autoantibodies that can target tissues. In some embodiments, the subject method can alter the activities or numbers of these cells involved in various autoimmune reactivities. In some embodiments, a subject method is effective to reduce the number and/or activity of an autoreactive cell in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive cells in the individual not treated with the HERV polypeptide.

In some embodiments, a subject method is effective to reduce the number and/or activity of an autoreactive T lymphocyte. Thus, in some embodiments, an effective amount of a HERV polypeptide is an amount that is effective to reduce the number and/or activity of autoreactive T lymphocytes in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive T lymphocytes in the individual not treated with the HERV polypeptide.

In some embodiments, a subject method is effective to reduce the number and/or activity of an autoreactive B cell. Thus, in some embodiments, an effective amount of a HERV polypeptide is an amount that is effective to reduce the number and/or activity of autoreactive B cells in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive B cells in the individual not treated with the HERV polypeptide.

Activities of an autoreactive T lymphocyte include, but are not limited to, cytolytic activity toward a “self” cell; secretion of cytokine(s); secretion of chemokine(s); responsiveness to chemokine(s); and trafficking. In some embodiments, an effective amount of a HERV polypeptide is an amount that is effective to reduce one or more activities of an autoreactive T lymphocyte in an individual.

Whether a HERV polypeptide is effective to reduce the number and/or activity of an autoreactive T lymphocyte in an individual is readily determined using known assays. For example, where the autoreactive T lymphocytes are specific for an autoantigen, the number and activity level of autoantigen-specific T lymphocytes is determined using, e.g., a mixed lymphocyte reaction in which irradiated cells comprising a detectable label in the cytoplasm and displaying the autoantigen are mixed with lymphocytes from the individual. Release of detectable label from the cytoplasm of the autoantigen-displaying cells indicates the presence in the individual of autoreactive lymphocytes. Methods of detecting autoreactive T lymphocytes associated with Type 1 diabetes are known in the art; and any such methods can be used. See, e.g., U.S. Pat. No. 6,022,697 for a discussion of a method of detecting autoreactive T lymphocytes associated with Type 1 diabetes.

In some embodiments, an effective amount of a HERV polypeptide is an amount that is effective to reduce the severity of one or more symptoms of an autoimmune disease. For example, in some embodiments, an effective amount of a HERV polypeptide is an amount that is effective to reduce the severity of one or more symptoms of an autoimmune disease by at least about 5%, at least about 10%, at least about 25%, at least about 30%, at least about 40%, at least about 50%, at least about 60%, at least about 70%, at least about 80%, or at least about 90%, or more, when compared to the severity of the symptom in an individual not treated with the HERV polypeptide.

Symptoms associated with autoimmune disorders are known in the art. See, e.g., “Textbook of the Autoimmune Diseases” R. G. Lahita, Ed. (2000) Lippincott Williams & Wilkins, 1^{st} ed. The following are non-limiting examples.

Multiple sclerosis is characterized by various symptoms and signs of central nervous system (CNS) dysfunction, with remissions and recurring exacerbations. The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances.

Diabetes Mellitus is syndrome characterized by hyperglycemia resulting from absolute or relative impairment in insulin secretion and/or insulin action. Although it may occur at any age, type I DM most commonly develops in childhood or adolescence and is the predominant type of DM diagnosed before age 30. This type of diabetes accounts for 10 to 15% of all cases of DM and is characterized clinically by hyperglycemia.

### Combination Therapies

In some embodiments, a subject treatment method will involve administering to an individual in need thereof an effective amount of a HERV polypeptide; and at least one additional agent that is effective for the treatment of an autoimmune disorder. In some embodiments, the at least one additional agent is other than a HERV polypeptide.

Those skilled in the art are aware of agents (other than a HERV polypeptide) that are suitable for treating autoimmune disorders. For example, agents that are suitable for treating Type 1 diabetes include insulin, including naturally occurring insulin, insulin analogs, and the like.

Insulin that is suitable for use herein includes, but is not limited to, regular insulin, semilente, NPH, lente, protamine zinc insulin (PZI), ultralente, insuline glargine, insulin aspart, acylated insulin, monomeric insulin, superactive insulin, hepatoselective insulin, and any other insulin analog or derivative, and mixtures of any of the foregoing. Insulin that is suitable for use herein includes, but is not limited to, the insulin forms disclosed in U.S. Pat. Nos. 4,992,417; 4,992,418; 5,474,978; 5,514,646; 5,504,188; 5,547,929; 5,650,486; 5,693,609; 5,700,662; 5,747,642; 5,922,675; 5,952,297; and 6,034,054; and published PCT applications WO 00/121197; WO 09/010,645; and WO 90/12814. Insulin analogs include, but are not limited to, superactive insulin analogs, monomeric insulins, and hepatospecific insulin analogs.

### Methods of Treating Schizophrenia

The present invention further provides methods of treating schizophrenia, the methods generally involving administering to an individual in need thereof an effective amount of a HERV polypeptide.

In these embodiments, an “effective amount” of a HERV polypeptide is an amount that, when administering to an individual in need thereof in one or more doses, reduces at least one symptom of schizophrenia by at least about 10%, at least about 15%, at least about 20%, at least about 25%, at least about 30%, at least about 35%, at least about 40%, at least about 45%, at least about 50%, or more, compared to the level or severity of the symptom in the individual in the absence of treatment with the HERV polypeptide. Symptoms of schizophrenia are known in the art, and include, e.g., “positive” symptoms (e.g., delusions, hallucinations, disorganized speech, grossly disorganized or catatonic behavior); and “negative” symptoms (e.g., alogia, affective flattening, avolition).

### Formulations

A HERV polypeptide, as described above, can be formulated in any of a variety of ways for administration to an individual in need thereof. The present invention provides pharmaceutical formulations comprising a HERV polypeptide. Immunogenic compositions comprising a HERV polypeptide are described above. Additional formulations are described below.

A subject formulation comprising a HERV polypeptide generally includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylprrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, e.g., for use in inducing or enhancing an immune response to a lentivirus, a HERV polypeptide is formulated for vaginal delivery. A subject formulation for intravaginal administration is formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

### Dosages

The appropriate dosage of a HERV polypeptide that, when administered in one or multiple doses, has the desired effect (e.g., increases a T cell immune response to a lentivirus; increases an immune response to a cancer cell; reduces an autoimmune response; etc.), will vary, depending on various factors, but will generally be in the range of from about 1 μg to about 100 mg, e.g., from about 1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 10 mg, from about 10 mg to about 50 mg, or from about 50 mg to about 100 mg, administered in one dose or divided into multiple doses.

In some embodiments, the amount of HERV polypeptide per dose is determined on a per body weight basis. For example, in some embodiments, a HERV polypeptide is administered in an amount of from about 0.5 mg/kg to about 100 mg/kg, e.g., from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, from about 40 mg/kg to about 50 mg/kg per dose, from about 50 mg/kg to about 60 mg/kg, from about 60 mg/kg to about 70 mg/kg, from about 70 mg/kg to about 80 mg/kg, from about 80 mg/kg to about 90 mg/kg, or from about 90 mg/kg to about 100 mg/kg, or more than about 100 mg/kg.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, multiple doses of a HERV polypeptide are administered. The frequency of administration of a HERV polypeptide can vary depending on any of a variety of factors, e.g., severity of the symptoms, etc. For example, in some embodiments, a HERV polypeptide is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a HERV polypeptide, e.g., the period of time over which a HERV polypeptide is administered, can vary, depending on any of a variety of factors, e.g., patient response, etc. For example, a HERV polypeptide can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

Conventional and pharmaceutically acceptable routes of administration include intranasal, intramuscular, intratracheal, intratumoral, transdermal, subcutaneous, intradermal, topical application, intravenous, vaginal, nasal, and other parenteral routes of administration. Suitable routes of administration also include oral and rectal routes. Routes of administration may be combined, if desired, or adjusted depending upon the agent and/or the desired effect. The composition can be administered in a single dose or in multiple doses.

A subject HERV composition can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, vaginal, transdermal, subcutaneous, intramuscular, intraorbital, intracapsular, intraspinal, intrastemal, intratumoral, peritumoral, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

A subject HERV composition can also be delivered to the subject by enteral administration. Enteral routes of administration include, but are not necessarily limited to, oral and rectal (e.g., using a suppository) delivery.

A subject HERV composition can be delivered to mucosal tissue, e.g., to vaginal tissue, to rectal tissue, etc.

### Methods of Generating HERV-Specific CTLs

The present invention provides methods of generating a population of HERV-specific CD8⁺ T cells in vitro. The methods generally involve contacting a CD8⁺ T cell, or a precursor thereof, with a HERV polypeptide in association with an antigen-presenting platform, where the contacting is performed in vitro. The methods are useful for generating a population of HERV polypeptide-specific CD8⁺ T cells, which are in turn useful in methods of treating disorders such as lentivirus infection (e.g., HIV infection) and cancer.

In some embodiments, CD8⁺ T cells are obtained from an individual, and are contacted in vitro with a HERV polypeptide in association with an antigen-presenting platform. In some embodiments, a mixed population of cells that comprises CD8⁺ T cells is obtained from an individual; and CD8⁺ T cells are isolated from the mixed population, generating an unstimulated CD8⁺ T cell population. The unstimulated CD8⁺ T cell population is then contacted in vitro a HERV polypeptide in association with an antigen-presenting platform. The contacting step activates at least a portion of the unstimulated CD8⁺ T cell population to become specific for a HERV polypeptide.

The source of the mixed cell population that comprises a CD8⁺ T cell can be, e.g., whole blood. The mixed cell population can manipulated in one or more ways or steps, e.g., to remove red blood cells; to select for CD8⁺ T cells; and/or to select against CD4⁺ T cells or other non-CD8⁺ cell populations. The number of unstimulated CD8⁺cells can range from about 10² cells to about 10⁹ cells, e.g., from about 10² cells to about 10³ cells, from about 10³ cells to about 10⁴ cells, from about 10⁴ cells to about 10⁵ cells, from about 10⁵ cells to about 5×10⁵ cells, from about 5×10⁵ cells to about 10⁶ cells, from about 10⁶ cells to about 5×10⁶ cells, from about 5×10⁶ cells to about 10⁷ cells, from about 10⁷ cells to about 5×10⁷ cells, from about 5×10⁷ cells to about 10⁸ cells, from about 10⁸ cells to about 5×10⁸ cells, or from about 5×10⁸ cells to about 10⁹ cells.

The antigen-presenting platform can be an antigen-presenting cell (APC), e.g., an APC pulsed with a HERV peptide, where the APC can be live or can be inactivated. In some embodiments, the antigen-presenting platform is a bead (e.g., a plastic bead, a magnetic bead, etc.), or other particle, to which a HERV peptide is bound. Antigen-presenting platforms other than naturally-occurring APCs are known in the art and include, but are not limited to, beads; inactivated surface-engineered viruses (see, e.g., Mosca et al. (2007) Retrovirol. 4:32); artificial APCs, e.g., liposomes (see, e.g., U.S. Patent Publication No. 2006/0034865); and the like.

The antigen-presenting platform will include, in addition to a HERV peptide, one or more surface molecules sufficient for stimulating expansion of a HERV-specific CD8⁺ T cell population, e.g., MHC class 1 molecules (e.g., HLA Class 1 molecules), etc. The antigen-presenting platform can also include one or more co-stimulatory molecules, where suitable co-stimulatory molecules include, but are not limited to, an anti-CD28 antibody, an anti-CD49d antibody, and the like).

The unstimulated CD8⁺ T cells are contacted in vitro with a HERV peptide in association with an antigen-presenting platform; and the number of HERV-specific CD8⁺ T cells is increased. The method results in a 10-fold to a 10⁶-fold increase in the number of HERV-specific CD8⁺ T cells. The number of HERV-specific CD8⁺ cells obtained by a subject method can range from about 10³ to about 10⁹ cells, e.g., from about 10³ cells to about 10⁴ cells, from about 10⁴ cells to about 10⁵ cells, from about 10⁵ cells to about 5×10⁵ cells, from about 5×10⁵ cells to about 10⁶ cells, from about 10⁶ cells to about 5×10⁶ cells, from about 5×10⁶ cells to about 10⁷ cells, from about 10⁷ cells to about 5×10⁷ cells, from about 5×10⁷ cells to about 10⁸ cells, from about 10⁸ cells to about 5×10⁸ cells, or from about 5×10⁸ cells to about 10⁹ cells.

The present invention provides treatment methods using the HERV-specific CD8⁺ T cells. In some embodiments, the methods are methods of treating an HIV infection. In other embodiments, the methods are methods of treating cancer. The methods generally involve administering to an individual in need thereof an effective amount of HERV-specific CD8⁺ T cells. In some embodiments, the HERV-specific CD8⁺ T cells are autologous, e.g., the HERV-specific CD8⁺ T cells are administered to the same individual from which the mixed cell population was obtained (i.e., the donor individual and the recipient individual are the same). In other embodiments, the HERV-specific CD8⁺ T cells are allogeneic, e.g., the HERV-specific CD8⁺ T cells are administered to an individual (a recipient individual) not genetically identical to the individual from which the mixed cell population was obtained (the donor individual).

In some embodiments, the HERV-specific CD8⁺ T cells are administered to a recipient individual in an amount of from about 10³ to about 10⁹ cells, e.g., from about 10³ cells to about 10⁴ cells, from about 10⁴ cells to about 10⁵ cells, from about 10⁵ cells to about 5×10⁵ cells, from about 5×10⁵ cells to about 10⁶ cells, from about 10⁶ cells to about 5×10⁶ cells, from about 5×10⁶ cells to about 10⁷ cells, from about 10⁷ cells to about 5×10⁷ cells, from about 5×10⁷ cells to about 10⁸ cells, from about 10⁸ cells to about 5×10⁸ cells, or from about 5×10⁸ cells to about 10⁹ cells, in one or more doses.

### Diagnostic Methods

The present invention provides various diagnostic methods, which methods utilize a subject HERV polypeptide or a subject HERV composition. Subject diagnostic methods include methods for monitoring a patient's response to treatment; methods for staging a disease; and methods for detecting a disease.

In some embodiments, a subject diagnostic method involves detecting the presence in an individual of a cancer cell that produces a HERV polypeptide. Methods for detecting a cancer cell that produces a HERV polypeptide include immunological methods, e.g., use of an antibody specific for a HERV polypeptide, where immunological assays include, e.g., immunohistological assays, and fluorescence activated cell analysis assays (e.g., fluorescence activated cell sorting assays, using a fluorescently labeled antibody to a HERV polypeptide).

In other embodiments, a subject diagnostic method generally involves detecting the number of HERV-specific CD8⁺ T cells in a biological sample obtained from an individual. The number of HERV-specific CD8⁺ T cells can be determined using, e.g., a ⁵¹Cr release assay, where target cells pulsed with a HERV peptide and labeled with ⁵¹Cr are contacted with a test sample that may contain HERV-specific CD8⁺ T cells. The number of HERV-specific CD8⁺ T cells is determined by measuring release of ⁵¹Cr from the target cells.

In other embodiments, a subject diagnostic method involves detecting a HERV polypeptide in the serum or plasma (or other biological fluid) of an individual. Detection of a HERV polypeptide in a biological fluid obtained from an individual can be carried out using, e.g., immunological assays employing antibody specific for a HERV polypeptide. Suitable immunological assays include, but are not limited to, enzyme-linked immunosorbent assays (ELISA), radioimmunoassays (RIA), protein blot (“Western blot”) assays, immunoprecipitation assays, and the like.

### HERV-Specific Antibodies

As noted above, in some embodiments, a subject diagnostic assay will employ an antibody specific for a HERV polypeptide (an “anti-HERV antibody”). Suitable anti-HERV antibodies include whole antibody of any isotype; epitope-binding fragments of an anti-HERV antibody; polyclonal antibodies; monoclonal antibodies; artificial antibodies; single-chain antibodies; and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies include mouse, rat, hamster, guinea pig, rabbit, etc. The antibody may be purified from the hybridoma cell supernatants or ascites fluid by conventional, techniques, e.g. affinity chromatography using protein bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Suitable anti-HERV antibodies also include “artificial” antibodies, e.g., antibodies and antibody fragments produced and selected in vitro. In some embodiments, such antibodies are displayed on the surface of a bacteriophage or other viral particle. In many embodiments, such artificial antibodies are present as fusion proteins with a viral or bacteriophage structural protein, including, but not limited to, M13 gene III protein. Methods of producing such artificial antibodies are well known in the art. See, e.g., U.S. Pat. Nos. 5,516,637; 5,223,409; 5,658,727; 5,667,988; 5,498,538; 5,403,484; 5,571,698; and 5,625,033.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

An anti-HERV antibody will in some embodiments be detectably labeled, e.g., with a radioisotope, an enzyme which generates a detectable product, a fluorescent protein, a chromogenic protein, and the like. An anti-HERV antibody may be further conjugated to other moieties, such as members of specific binding pairs, e.g., biotin (member of biotin-avidin specific binding pair), and the like. An anti-HERV antibody may also be bound to a solid support, including, but not limited to, polystyrene plates or beads, magnetic beads, test strips, membranes, and the like.

An antibody specific for a HERV polypeptide can be labeled, directly or indirectly. Direct labels include radioisotopes (e.g., ¹²⁵I; ³⁵S, and the like); enzymes whose products are detectable (e.g., luciferase, β-galactosidase, horse radish peroxidase, alkaline phosphatase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin; fluorescent proteins (e.g., a green fluorescent protein, a yellow fluorescent protein, etc.); and the like. Indirect labels include second antibodies specific for HERV-specific antibodies, wherein the second antibody is labeled as described above; and members of specific binding pairs, e.g., biotin-avidin, and the like.

In some embodiments, an anti-HERV antibody comprises, covalently linked to the antibody, a protein that provides for a detectable signal. Suitable proteins include, but are not limited to, fluorescent proteins and enzymes (e.g., β-galactosidase, luciferase, horse radish peroxidase, alkaline phosphatase, etc.). Suitable fluorescent proteins include, but are not limited to, a green fluorescent protein (GFP), including, but not limited to, a GFP derived from Aequoria victoria or a derivative thereof, a number of which are commercially available; a GFP from a species such as Renilla reniformis, Renilla mulleri, or Ptilosarcus guernyi, as described in, e.g., WO 99/49019 and Peelle et al. (2001) J. Protein Chem. 20:507-519; any of a variety of fluorescent and colored proteins from Anthozoan species, as described in, e.g., Matz et al. (1999) Nature Biotechnol. 17:969-973, U.S. Patent Publication No. 2002/0197676, or U.S. Patent Publication No. 2005/0032085; and the like.

### Monitoring Patient Response to Treatment for a Lentivirus Infection

In some embodiments, a subject HERV polypeptide composition is useful for monitoring a patient's response to treatment for a lentivirus infection, e.g., an HIV infection. Thus, the present invention further provides methods for monitoring a patient's response to treatment for a lentivirus infection, e.g., an HIV infection. The methods generally involve contacting a white blood cell (WBC) from a patient in vitro with a subject HERV polypeptide; and detecting a cytokine secreted by the WBC in response to contact with the HERV polypeptide. A reduction in cytokine production by the WBC in response to contact with a HERV polypeptide is an indication that the treatment is effective in treating a lentivirus infection (e.g., in achieving a reduction in viral load, in achieving an increase in CD4⁺ T lymphocyte levels (in the case of an HIV infection), and the like). Suitable WBC include, but are not limited to, peripheral blood mononuclear cells (PBMC), isolated T lymphocytes, isolated CD4⁺ T lymphocytes, isolated CD8⁺ T lymphocytes, natural killer (NK) cells, natural killer T lymphocytes (NKT, e.g., NK1.1⁺ T lymphocytes), and the like.

HERV polypeptides suitable for use in a subject monitoring method can be 9 amino acids, 10 amino acids, 11 amino acids, 12 amino acids, 12-15 amino acids, 15-18 amino acids, 18-20 amino acids, or 20-25 amino acids long, or longer. Suitable HERV polypeptides include any of the HERV polypeptides discussed above. In some embodiments, the HERV polypeptide comprises an amino acid sequence as set forth in any one of SEQ ID NOs: 1-25.

Cytokines that are secreted from PBMC and that are detected in a subject patient monitoring method include, but are not limited to, IFN-γ, TNF-α, and IL-2.

Methods for detecting secreted cytokines that are suitable for use in a subject patient monitoring method include, but are not limited to, immunological assays, e.g., enzyme-linked immunosorbent assay (ELISA), radioimmunoassay (RIA), an enzyme-linked immunospot (ELISPOT) assay; cellular assays; and the like.

In some embodiments, a reduction of at least about 10%, at least about 20%, at least about 30%, at least about 40%, at least about 50%, at least about 60%, at least about 70%, at least about 80%, or at least about 90% or more, in cytokine production by WBC in response to contact with a HERV polypeptide indicates that the treatment for the lentivirus infection is efficacious.

Patient samples comprising WBC can be obtained before and after treatment; or at various times during the course of treatment, and the level of cytokine production compared between a sample taken at a first time point and a sample taken at a second (later) time point.

In some embodiments, PBMC obtained from a patient are contacted with one or more HERV polypeptides in vitro; and an ELISPOT assay is used to detect cytokine production. The ELISPOT assay has been described in the art. See, e.g., Lalvani et al. (1997) J. Exp. Med. 186:859; and U.S. Pat. No. 5,853,697. In these embodiments, the level of cytokines produced by the PBMC is expressed as the number of spot-forming units (SFU) per 10⁶ PBMC. A reduction in the number of SFU indicates that a treatment for a lentivirus infection is effective.

### Monitoring Patient Response to Cancer Treatment

The present invention provides methods of monitoring patient response to a treatment regimen for cancer. The level of a HERV polypeptide associated with the cancer is monitored, before, during a treatment regimen, and after a treatment regimen.

In some embodiments, the level of a HERV polypeptide is monitored, e.g., in serum, on the surface of a particular cell population, etc.

### Staging a Disease

The present invention provides methods of staging a disease in an individual, where the level of a HERV polypeptide is associated with the stage or severity of the disease. The methods generally involve detecting the level of a HERV polypeptide in a biological sample obtained from the individual. The level of the HERV polypeptide in the biological sample is correlated with the severity of the disease or disorder, and used to stage the disease.

In some embodiments, a subject method of staging a disease involves detecting the number of CD8⁺ T cells, in a biological sample obtained from an individual, that are specific for a subject HERV polypeptide. In some embodiments, the number of HERV-specific CD8⁺ T cells is an indication of the stage of the disease.

### Detecting a Disease

The present invention provides methods of detecting a disease such as a cancer in an individual, where the presence or level of a HERV polypeptide in a biological sample obtained from the individual indicates the presence of a cancerous cell in the biological sample (and hence the individual). The methods generally involve detecting the level of a HERV polypeptide in a biological sample obtained from the individual. Where the level of the HERV polypeptide is higher than the level associated with a normal cell, such is an indication of the presence in the sample of a cancerous cell.

### Subjects Suitable for Treatment

### Treatment of Lentivirus Infection

The methods of the present invention are suitable for treating individuals who have a lentiviral infection; uninfected individuals who are at risk of contracting a lentiviral infection; individuals who were treated for a lentiviral infection, but failed to respond to the treatment; and individuals who were treated for a lentiviral infection, but who relapsed.

For example, the methods of the present invention are suitable for treating individuals who have a human immunodeficiency virus (HIV) infection; individuals who are naïve with respect to HIV infection, but who at risk of contracting an HIV infection; and individuals who were treated for an HIV infection, but who either failed to respond to the treatment, or who initially responded to treatment but subsequently relapsed. Such individuals include, but are not limited to, uninfected individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals; intravenous drug users; individuals who may have been exposed to HIV-infected blood, blood products, or other HIV-contaminated body fluids; and babies who are being nursed by HIV-infected mothers. Individuals suitable for treatment include individuals infected with, or at risk of becoming infected with, HIV-1 and/or HIV-2 and/or HIV-3, or any variant thereof.

### Treatment of HTLV Infection

The above-described methods can be used to treat a human T cell leukemia virus (HTLV) infection in an individual, e.g., an HTLV-I or HTLV-II infection. Thus, a subject method is also suitable for treating individuals who have been infected with an HTLV; individuals who have not yet been infected with HTLV, but who are at risk of becoming infected with HTLV; and individuals who have not yet been infected with HTLV, but who may in the future become infected with HTLV.

### Cancer Treatment

The methods of the present invention are suitable for treating individuals diagnosed with a cancer associated with expression of HERV, where such cancers include, but are not limited to, breast cancer, ovarian cancer, melanoma, teratoma, seminoma, prostate cancer, and testicular cancer. The methods of the present invention are suitable for treating individuals who have been diagnosed with breast cancer; individuals who have been diagnosed with ovarian cancer; and individuals who have been diagnosed with testicular cancer. A subject method of treating cancer is also suitable for treating individuals who have been treated for breast cancer, ovarian cancer, melanoma, teratoma, seminoma, prostate cancer, or testicular cancer, and who either failed to respond to the treatment, or responded initially, then relapsed.

### Treatment of an Autoimmune Disorder

The methods of the present invention are suitable for treating individuals diagnosed with an autoimmune disorder, where such autoimmune disorders include, but are not limited to, multiple sclerosis, rheumatoid arthritis, systemic lupus erythematosus, and Type 1 diabetes. The methods of the present invention are suitable for treating individuals who have been treated for an autoimmune disorder, and who either failed to respond to the treatment, or responded initially, then relapsed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### HERV Peptides Stimulate Cytokine Production in Human PBMCs

### Materials and Methods

Patients.

HIV-1 positive volunteers were selected for this study. The study was approved by the local institutional review board and subjects were given written informed consent. Studies were performed on cryopreserved PBMC from various patient timepoints.

Peptide Selection.

Selection of candidate HERV epitopes was based on translated HERV protein sequence data compiled from publicly available databases. HIV-1 peptides were designed from the sequences of known HIV-1 epitopes listed in the Los Alamos National Laboratory HIV immunology database. Antigenic regions of HERV insertions were assigned an HLA restriction with epitope prediction software [SYFPEITHI²⁹; SEQ ID NO:36] or based on the HLA restriction of the corresponding HIV-1 epitope.

ELISPOT Assay.

ELISPOT analysis was performed as previously described³⁴. Plates were incubated 15-18 hours at 37° C. Equivalent antigen concentrations were used for HIV and HERV peptide response comparisons. Assays were performed with duplicate wells for each condition, expect where cell recovery from archived samples dictated the use of single wells. Plates were counted with an AID ELISPOT reader (Cell Technology,). Spot totals for duplicate wells were averaged, and all spot numbers were normalized to numbers of IFN-γ spot-forming units (SFU) per 1×10⁶PBMC. Spot values from media control wells were subtracted to determine responses to each peptide. Any resulting peptide values <0 following media subtraction were set to 0 for further analysis.

HERV-K Expression Detection.

Expression levels of a HERV-K derived envelope transcript³⁵ were measured in HIV-1 positive 1 ml plasma samples and HIV-1 negative low-risk controls. Plasma samples were centrifuged at low speed and filtered prior to RNA collection to remove remaining cellular contaminants. High speed centrifugation was used to pellet particles for RNA isolation with Trizol reagent (Invitrogen). Samples were pre-treated with DNAse to eliminate genomic DNA contamination as a source of amplified HERV sequences. RT-PCR was performed on samples along with control amplifications without RT enzyme. As a calibration standard, cellular transcript expression of HERV and the housekeeping control gene β-actin was measured in cDNA prepared from 2.5×10⁶ HIV-negative donor PBMCs. Quantification standards were also prepared by serial dilution of the cellular cDNA. Quantitative PCR with primers specific for the transcripts of interest was performed on all samples with the ABI Prism 7900HT Sequence Detection System (Applied Biosystems) using SYBR-Green detection. Expression levels are presented as percentages relative to PBMC derived standards, and represent the means of triplicate reactions. Gel electrophoresis and melting point analysis of PCR products were used to confirm product purity and accurate amplicon size.

### ⁵¹Cr Release Assays

Cryopreserved peripheral blood mononuclear cells (PBMC) from a study participant who responded to the HERV-L IQ10 peptide were stimulated for 7 days with peptides or pools of each antigen. Autologous, irradiated, peptide-pulsed feeder cells were used to restimulate after 7 days. Cells were tested for their ability to lyse peptide-pulsed, autologous, EBV-transformed B cell lines by measuring the percentage of specific ⁵¹Cr release.

### Results

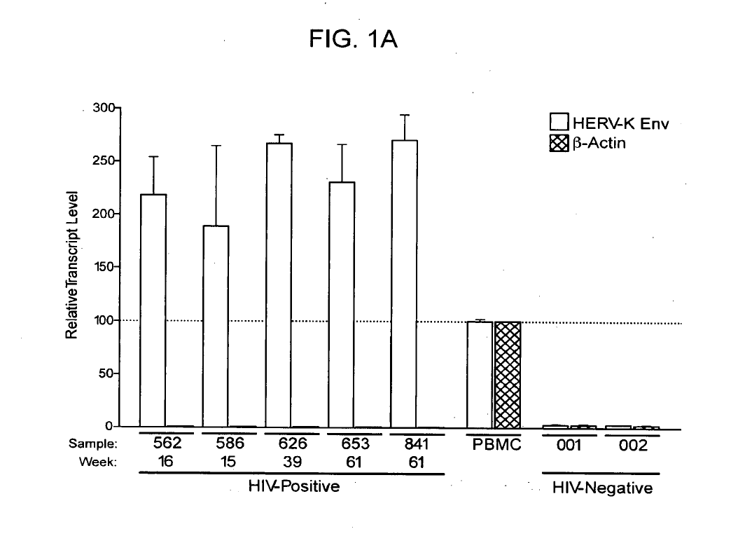

To identify differences between expression levels of HERVs in HIV-1 positive and negative subjects, an RT-PCR analysis was performed on plasma to quantify a transcript derived from the youngest family of endogenous retroviruses in the human genome, HERV-K (FIG. 1A). Expression of the HERV-K transcript was detected in HIV-1 positive plasma, but not in HIV-1 negative controls. The amount of HERV-K transcripts in the plasma of HIV-1 positive individuals was greatly out of proportion to that of other non virion-associated cellular transcripts (β-actin), thus ruling out cellular debris as an etiology for these transcripts. Data from additional individuals are presented in FIG. 1B, which shows plasma RNA levels of HERV-K in HIV-1-positive and HIV-1-negative individuals' plasma.

FIGS. 1A and 1B.

Expression of HERV-K transcripts in HIV positive and negative individuals' plasma a, Levels of a HERV-K transcript derived from the envelope region measured relative to levels detected in peripheral blood cells (set to a value of 100 for comparison) shown as unfilled bars. Levels of a cellular control gene (β-actin) are shown as filled bars. Levels of the control gene measured in peripheral blood cells were also set to 100 for relative comparison to other samples b, Levels of the HERV-K transcript measured in the plasma of HIV-1 positive (filled circles) and HIV-1-negative individuals (open circles).

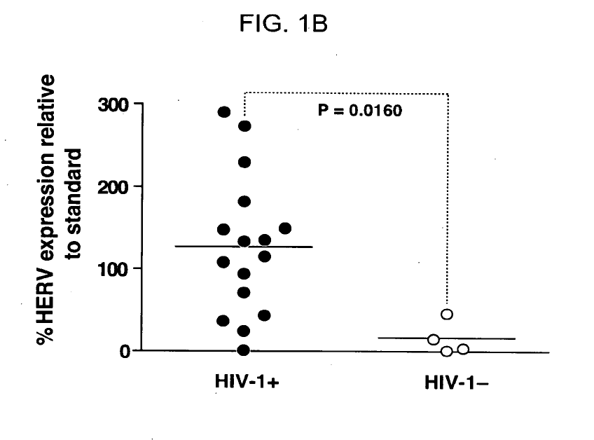

When HERVs are expressed, the potential exists to generate an immune response against these antigens. Given that these are also endogenous antigens, it is unclear whether the response will be immunogenic or tolerogenic in nature. It was hypothesized that in regions of HIV-1 that are highly similar to HERVs, tolerance to HERVs could impair the HIV-1-specific immune response. Cross-tolerance has recently been suggested as a mechanism hampering the body's ability to produce antibodies that neutralize HIV-1 due to their cross-reactivity with a self-antigen cardiolipin¹⁸. Although HIV-1 and endogenous retroviruses are phylogenetically distant¹⁹, the similarity between them was analyzed from the perspective of a T cell receptor, focusing on short regions of high similarity corresponding to the length of T cell epitopes (8-12 amino acids). These regions of similarity are typically rejected in standard phylogenetic analysis, as they are small enough to occur frequently by chance, without indicating any genetic relatedness. Because the T cell recognizes proteins in short peptides presented on HLA molecules, these regions of similarity have significance for the immune response (FIG. 2). Since reverse transcriptase is a highly conserved protein, we expected and observed both clustered and distributed amino acid identity. Less conserved proteins such as Gag showed primarily clustered amino acid identities.

FIG. 2.

HERV/HIV amino acid alignments of HIV HXB-2 and various HERV insertions (identified by their HERVd²⁸ or NCBI accession number) showing segments of the Gag and Reverse Transcriptase proteins. Identical amino acids are shown in boxes. Alignments were anchored based on short regions of similarity identified with BLAST³⁶ short nearly exact match search settings, which included both amino acid similarity (not shown in this figure) and identity.

Thirty-one HIV-1 positive volunteers and five low-risk HIV-1 negative controls were screened by ELISPOT for responses to a panel of peptides derived from HERV insertions and HIV-1 proteins (Table 1) with varying levels of amino acid sequence identity to each other.

TABLE 1
HERV and HIV-1-derived peptide data

| | Epitope | Percent | Sequence | HLA | Start | | Sequence |
|---|---|---|---|---|---|---|---|
| Peptide Name | Alignment | Identity | Identifier | restriction | Position | Accession | Origin |
| not tested HERV-K FD10 | 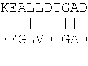 | 70% | SEQ ID NO: 39 SEQ ID NO: 12 | No known epitope | Protease: 20 | gi52001472 | HXB-2 HERV-K |
| HERV Gag KK9 HERV-L KF9 | 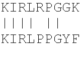 | 67% | SEQ ID NO: 40 SEQ ID NO: 8 | A3/B27 | Gag p17: 18 | 162613 | HXB-2 HERV-L |
| not tested HERV-L SF9 | 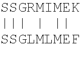 | 67% | SEQ ID NO: 41 SEQ ID NO: 7 | No known epitope | gp160: 143 | 162605 | HXB-2 HERV-L |
| HIV RT TI9 HERV-K FI8 | 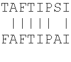 | 67% | SEQ ID NO: 42 SEQ ID NO: 10 | B51 | RT p51: 128 | gi5802821 | HXB-2 HERV-K |
| HIV RT IL9 HERV-K VR9 | 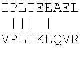 | 44% | SEQ ID NO: 43 SEQ ID NO: 14 | B35 | RT p51: 293 | | HXB-2 HERV-K |
| HIV Gag SL9 HERV-L PL9 | 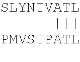 | 44% | SEQ ID NO: 44 SEQ ID NO: 4 | A2 | Gag p17:77 | 162568 | HXB-2 HERV-L |
| HIV gp160 SY9 HERV-C TE9 | 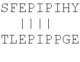 | 44% | SEQ ID NO: 45 SEQ ID NO: 16 | | gp160: 209 | | HXB-2 HERV-C |
| not tested HERV-K FI9 | 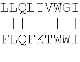 | 44% | SEQ ID NO: 46 SEQ ID NO: 13 | A2 | gp160: 565 | Rakoff- Nahoum | HXB-2 HERV-K |
| not tested HERV-K GQ9 | 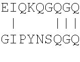 | 44% | SEQ ID NO: 47 SEQ ID NO: 11 | No known epitope | RT p51: 328 | gi67782351 | HXB-2 HERV-K |
| HIV Nef LG13 HERV-H LI12 | 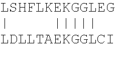 | 46% | SEQ ID NO: 48 SEQ ID NO: 15 | B35 | Nef: 87 | | HXB-2 HERV-H |
| HIV RT VL9 HERV-L II9 | 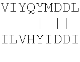 | 33% | SEQ ID NO: 49 SEQ ID NO: 2 | A2 | RT p51: 179 | 162563 | HXB2 HERV-L |
| HIV RT NY9 HERV-L LY9 | 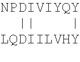 | 33% | SEQ ID NO: 50 SEQ ID NO: 3 | B35 | RT p51: 175 | 162563 | HXB-2 HERV-L |
| not tested HERV-W DK10 | 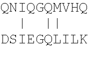 | 33% | SEQ ID NO: 51 SEQ ID NO: 9 | multiple | Gag p24: 4 | gi52000737 | HXB-2 HERV-W |
| HIV RT TY9 HERV-L AF9 | 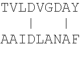 | 22% | SEQ ID NO: 52 SEQ ID NO: 5 | B35 | RT p51: 107 | 162604 | HXB2 HERV-L |
| HIV RT VY10 HERV-L IQ10 | 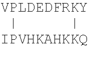 | 20% | SEQ ID NO: 53 SEQ ID NO: 6 | B35 | RT p51: 118 | 162604 | HXB2 HERV-L |
| HIV RT RG10 HERV-L SL10 | 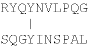 | 11% | SEQ ID NO: 54 SEQ ID NO: 1 | B51 | RT p51: 143 | 162563 | HXB-2 HERV-L |

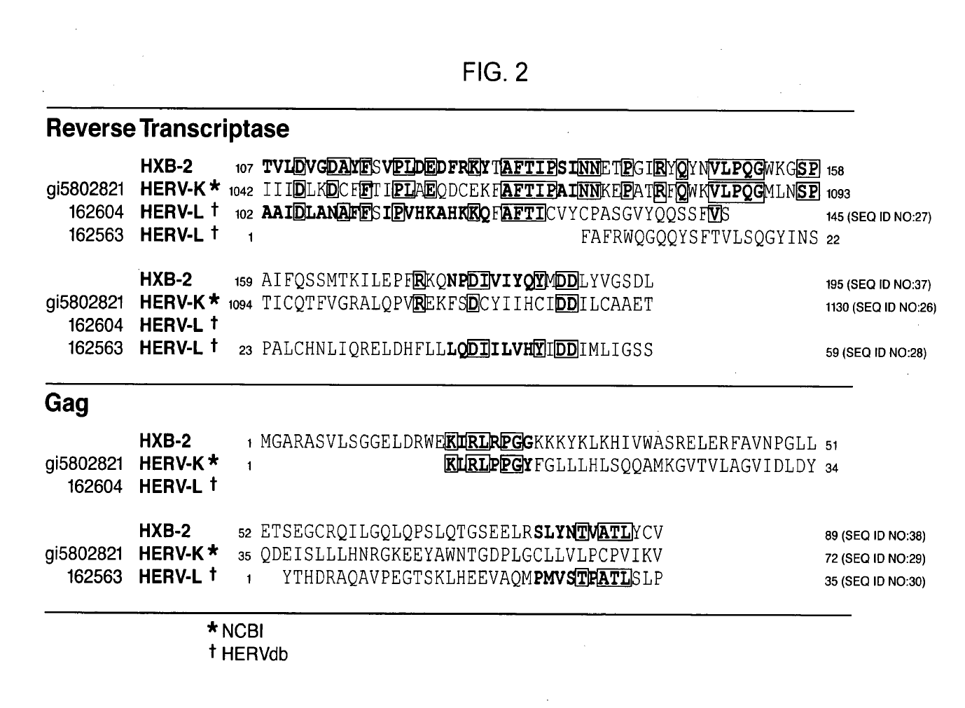

Strong interferon gamma specific T cell responses were detected to HERV peptides in HIV-1 infected volunteers but not in HIV-1 negative controls (FIG. 3, Mann-Whitney, P<0.05). The magnitude of the HIV-1 T cell response was directly associated with the magnitude of the HERV T cell response.

FIG. 3.

T cell responses to HERV and HIV-1 antigens in 29 HIV-1 positive and 13 low-risk HIV-1 negative individuals measured by Interferon-gamma ELISPOT. HERV peptides were grouped according to their similarity to HIV-1 peptide sequence, with ‘Unique HERV Peptides’ having 3 or less amino acids in common with an HIV-1 peptide, and ‘HERV Peptides similar to HIV-1’ having 4 or more peptides in common with HIV-1. Subsets of peptides were tested in each patient, with the number tested (n=6-23) varying depending on HLA type. Values shown for responses are normalized per peptide within each grouping (i.e. the sum of the response values to all peptides tested divided by the number of peptides tested for each patient). Responses in HIV-1 positive individuals are shown as closed circles and in HIV-1 negative individuals are shown as open circles. Responses to all HERV peptides were measured for HCV+ individuals and are shown as filled triangles. P-values are derived from the Mann-Whitney test.

As this association could indicate HIV-1 specific T cells cross-reacting on HERV antigens, the frequency of responses for each HERV peptide and its counterpart HIV-1 peptide was compared. For each HIV-1/HERV peptide pairing, there were variable numbers of amino acids in common between the two peptides. High frequency HERV peptide responses were detected at low levels of amino acid identity to HIV-1 peptides, indicating that HERV-specific responses are generated independently. It was concluded that cross-reactive HIV-1-specific T cells cannot be solely responsible for the responses against HERVs observed.

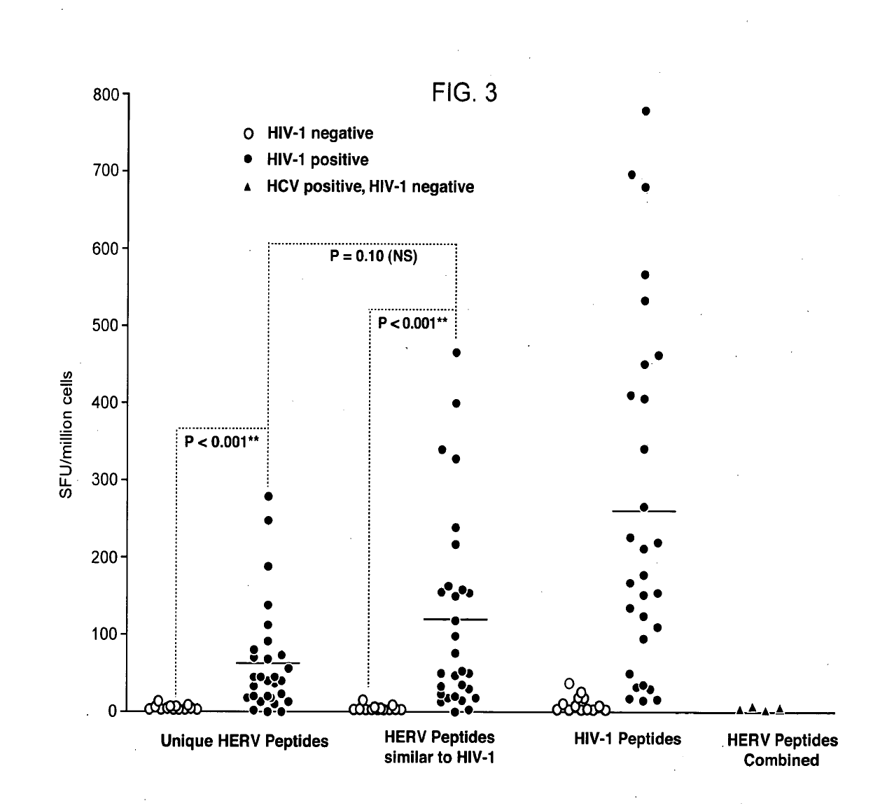

FIG. 4 depicts an inverse correlation between anti-HERV T cell responses and HIV-1 plasma viral load. PBMC from twenty HIV-1+ individuals not on treatment were analyzed by ELISPOT for HERV responses. The mean response (>50 SFU/million PBMC) values for all HERV peptides tested had a significant inverse correlation to HIV-1 plasma viral load (Spearman, two-tailed, r=−0.49, P=0.03) and by linear regression (r²=0.39, P=0.003) as shown in the figure.

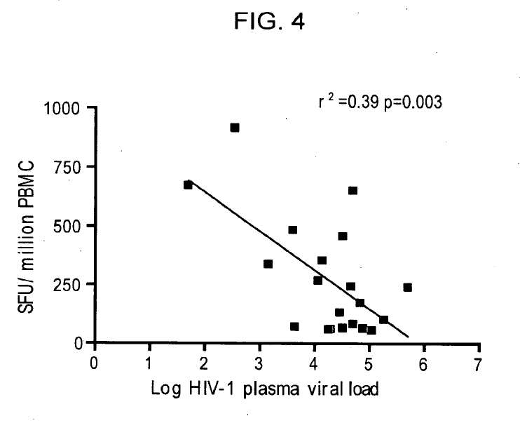

Because the ability to control viral load by eliminating infected cells depends on killing, the ability of HERV specific CD8+ T cells to kill autologous B cells presenting their target peptide was measured. PBMC from one subject (OP841) were peptide stimulated to enrich for responsive CD8⁺ T cells. After a two-week peptide stimulation, the ⁵¹Cr-release assay was used to measure the ability of the enriched CD8⁺ T cells to kill EBV-transformed B cell targets presenting cognate peptide. CD8⁺ T cells enriched by stimulation with HERV peptide were able to kill B cell targets presenting their cognate peptide but did not lyse targets loaded with a non-cognate or no peptide (FIG. 5).

FIG. 5 depicts ⁵¹Cr release from target cells. HERV-L IQ10-specific T cells were tested against autologous B cells pulsed with HERV-L IQ10 peptide (filled circles), control peptide (open circles) or no peptide (filled triangles).

The data demonstrate an elevation in HERV transcript expression and T cell responses directed at HERV peptides associated with HIV-1 infection. A naturally-arising T cell response against HERVs in HIV-1-infected individuals indicates the feasibility of inducing responses earlier in infection, or in at risk uninfected individuals, as a novel HIV-1 vaccine paradigm. One of the greatest difficulties in HIV-1 vaccine development is overcoming the mutability of the virus, which enables it to evade specific immune responses elicited with a vaccine. HERVs are genome-encoded elements; translation products produced from de-regulated transcription of HERV insertions is expected to be far less variable than HIV-1 proteins. If HERV antigen production and presentation is a consequence of HIV-1 infection of a cell, the HERV products serve as a stably recognizable surrogate marker signalling HIV-1 infection to the immune system. Educating the immune system to recognize the HERV surrogate marker through vaccination induces killing of HIV-1-infected cells, circumventing the need to recognize highly variable HIV-1 antigens.

### REFERENCES

1. Bannert, N. & Kurth, R. Retroelements and the human genome: new perspectives on an old relation. Proc Natl Acad Sci USA 101 Suppl 2, 14572-9 (2004).
2. Perl, A. Role of endogenous retroviruses in autoimmune diseases. Rheum Dis Clin North Am 29, 123-43, vii (2003).
3. Roelofs, H., van Gurp, R. J. H. L. M., Oosterhuis, J. W. & Looijenga, L. H. J. Detection of Human Endogenous Retrovirus Type K-Specific Transcripts in Testicular Parenchyma and Testicular Germ Cell Tumors of Adolescents and Adults: Clinical and Biological Implications. Am J Pathol 153, 1277-1282 (1998).
4. Seifarth, W. et al. Assessment of retroviral activity using a universal retrovirus chip. J Virol Methods 112, 79-91 (2003).
5. Yang, J. et al. An ancient family of human endogenous retroviruses encodes a functional homolog of the HIV-1 Rev protein. Proc Natl Acad Sci USA 96, 13404-8 (1999).
6. Esnault, C. et al. APOBEC3G cytidine deaminase inhibits retrotransposition of endogenous retroviruses. Nature 433, 430-433 (2005).
7. Esnault, C., Millet, J., Schwartz, 0. & Heidmann, T. Dual inhibitory effects of APOBEC family proteins on retrotransposition of mammalian endogenous retroviruses. Nucl. Acids Res. 34, 1522-1531 (2006).
8. Stopak, K., de Noronha, C., Yonemoto, W. & Greene, W. C. HIV-1 Vif Blocks the Antiviral Activity of APOBEC3G by Impairing Both Its Translation and Intracellular Stability. Molecular Cell 12, 591-601 (2003).
9. Sheehy, A. M., Gaddis, N. C. & Malim, M. H. The antiretroviral enzyme APOBEC3G is degraded by the proteasome in response to HIV-1 Vif. Nat Med 9, 1404-1407 (2003).
10. Lander, E. S. et al. Initial sequencing and analysis of the human genome. Nature 409, 860-921 (2001).
11. Best, S., Le Tissier, P., Towers, G. & Stoye, J. P. Positional cloning of the mouse retrovirus restriction gene Fv1. Nature 382, 826-9 (1996).
12. Nihrane, A., Fujita, K., Willey, R., Lyu, M. S. & Silver, J. Murine leukemia virus envelope protein in transgenic-mouse serum blocks infection in vitro. J Virol 70, 1882-9 (1996).
13. Blond, J. L. et al. Molecular characterization and placental expression of HERV-W, a new human endogenous retrovirus family. J Virol 73, 1175-85 (1999).
14. Blond, J. L. et al. An envelope glycoprotein of the human endogenous retrovirus HERV-W is expressed in the human placenta and fuses cells expressing the type D mammalian retrovirus receptor. J Virol 74, 3321-9 (2000).
15. Mi, S. et al. Syncytin is a captive retroviral envelope protein involved in human placental morphogenesis. Nature 403, 785-9 (2000).
16. Stauffer, Y., Theiler, G., Sperisen, P., Lebedev, Y. & Jongeneel, C. V. Digital expression profiles of human endogenous retroviral families in normal and cancerous tissues. Cancer Immun 4, 2 (2004).
17. Medstrand, P. & Blomberg, J. Characterization of novel reverse transcriptase encoding human endogenous retroviral sequences similar to type A and type B retroviruses: differential transcription in normal human tissues. J Virol 67, 6778-87 (1993).
18. Haynes, B. F. et al. Cardiolipin polyspecific autoreactivity in two broadly neutralizing HIV-1 antibodies. Science 308, 1906-8 (2005).
19. Coffin, J. M., Hughes, S. H., and Varmus, H. E. (eds.). Retroviruses (Cold Spring Harbor Laboratory Press, Cold Spring Harbor, N.Y., 1997).
20. Amdahl, E. M., Michaelsson, J., Moretto, W. J., Hecht, F. M. & Nixon, D. F. Human CD4+CD25+ Regulatory T Cells Control T-Cell Responses to Human Immunodeficiency Virus and Cytomegalovirus Antigens. J. Virol. 78, 2454-2459 (2004).
21. Rawal, B. D. et al. Development of a New Less-Sensitive Enzyme Immunoassay for Detection of Early HIV-1 Infection. Journal of Acquired Immune Deficiency Syndromes 33, 349-355 (2003).
22. Lindeskog, M., Medstrand, P. & Blomberg, J. Sequence variation of human endogenous retrovirus ERV9-related elements in an env region corresponding to an immunosuppressive peptide: transcription in normal and neoplastic cells. J Virol 67, 1122-6 (1993).
23. Schindler, M. et al. Nef-mediated suppression of T cell activation was lost in a lentiviral lineage that gave rise to HIV-1. Cell 125, 1055-67 (2006).
24. van Lier, R. A., ten Berge, I. J. & Gamadia, L. E. Human CD8(+) T-cell differentiation in response to viruses. Nat Rev Immunol 3, 931-9 (2003).
25. Champagne, P. et al. Skewed maturation of memory HIV-specific CD8 T lymphocytes. 410, 106-111 (2001).
26. Appay, V. et al. Memory CD8+ T cells vary in differentiation phenotype in different persistent virus infections. 8, 379-385 (2002).
27. Villesen, P., Aagaard, L., Wiuf, C. & Pedersen, F. S. Identification of endogenous retroviral reading frames in the human genome. Retrovirology 1, 32 (2004).
28. Paces, J. et al. HERVd: the Human Endogenous RetroViruses Database: update. Nucleic Acids Res 32, D50 (2004).
29. Hans-Georg Rammensee, J. B., Niels Nikolaus Emmerich, Oskar Alexander Bachor, Stefan Stevanovic. SYFPEITHI: database for MHC ligands and peptide motifs. (access via the world wide web at syfpeithi.de). Immunogenetics 50, 213-219 (1999).
30. Rakoff-Nahoum, S. et al. Detection of T Lymphocytes Specific for Human Endogenous Retrovirus K (HERV-K) in Patients with Seminoma. AIDS Research and Human Retroviruses 22, 52-56 (2006).
31. Jern, P., Sperber, G. O., Ahlsen, G. & Blomberg, J. Sequence variability, gene structure, and expression of full-length human endogenous retrovirus H. J Virol 79, 6325-37 (2005).
32. Flockerzi, A., Burkhardt, S., Schempp, W., Meese, E. & Mayer, J. Human endogenous retrovirus HERV-K14 families: status, variants, evolution, and mobilization of other cellular sequences. J Virol 79, 2941-9 (2005).
33. Macfarlane, C. & Simmonds, P. Allelic variation of HERV-K(HML-2) endogenous retroviral elements in human populations. J Mol Evol 59, 642-56 (2004).
34. Meiklejohn, D. A. et al. ELISPOT cell rescue. J Immunol Methods 288, 135-47 (2004).
35. Conrad, B. et al. A Human Endogenous Retroviral Superantigen as Candidate Autoimmune Gene in Type I Diabetes. Cell 90, 303-313 (1997).
36. Altschul, S. F. et al. Gapped BLAST and PSI-BLAST: a new generation of protein database search programs. Nucleic Acids Res 25, 3389-402 (1997).
37. Betts, M. R. et al. Sensitive and viable identification of antigen-specific CD8+ T cells by a flow cytometric assay for degranulation. Journal of Immunological Methods 281, 65-78 (2003).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-12. (canceled)

13. A method of inducing a T lymphocyte response in an individual to a host cell infected with a pathogenic virus, the method comprising administering to the individual:
a) an immunogenic composition comprising: i) a human endogenous retrovirus-K (HERV-K) polypeptide; and ii) a pharmaceutically acceptable carrier; or
b) an immunogenic composition comprising: i) a nucleic acid comprising a nucleotide sequence encoding a HERV-K polypeptide; and ii) a pharmaceutically acceptable carrier,
wherein said HERV-K polypeptide induces a T lymphocyte response in the individual to a host cell infected with a pathogenic virus.

14. The method of claim 13, wherein the T lymphocyte response comprises a CD8⁺ T cell response or a CD4⁺ T cell response.

15. The method of claim 13, wherein the T lymphocyte response comprises a mucosal T lymphocyte response.

16. The method of claim 13, wherein the pathogenic virus is a human immunodeficiency virus.

17. The method of claim 13, wherein the individual has not been infected with the pathogenic virus.

18. The method of claim 13, wherein the individual has been infected with the pathogenic virus.

19.-22. (canceled)

23. The method of claim 13, wherein said administration is parenteral administration.

24. The method of claim 13, wherein said administration is mucosal administration.

25. The method of claim 13, wherein said immunogenic composition comprises an adjuvant.

26. The method of claim 25, wherein the adjuvant comprises aluminum hydroxide, MF59, or monophosphoryl lipidA.

27. The method of claim 13, wherein the nucleic acid is a recombinant vector.

28. The method of claim 27, wherein the recombinant vector is a recombinant viral vector.

29. The method of claim 13, wherein the HERV-K polypeptide is a HERV-K env polypeptide, a HERV-K gag polypeptide, or a HERV-K pol polypeptide.


SEQUENCE LISTING (54 sequences)

SEQ ID NO: 1 - protein, 10 aa, Artificial Sequence
  synthetic peptide
SQGYINSPAL

SEQ ID NO: 2 - protein, 9 aa, Artificial Sequence
  synthetic peptide
ILVHYIDDI

SEQ ID NO: 3 - protein, 9 aa, Artificial Sequence
  synthetic peptide
LQDIILVHY

SEQ ID NO: 4 - protein, 9 aa, Artificial Sequence
  synthetic peptide
PMVSTPATL

SEQ ID NO: 5 - protein, 9 aa, Artificial Sequence
  synthetic peptide
AAIDLANAF

SEQ ID NO: 6 - protein, 10 aa, Artificial Sequence
  synthetic peptide
IPVHKAHKKQ

SEQ ID NO: 7 - protein, 9 aa, Artificial Sequence
  synthetic peptide
SSGLMLMEF

SEQ ID NO: 8 - protein, 9 aa, Artificial Sequence
  synthetic peptide
KIRLPPGYF

SEQ ID NO: 9 - protein, 10 aa, Artificial Sequence
  synthetic peptide
DSIEGQLILK

SEQ ID NO: 10 - protein, 8 aa, Artificial Sequence
  synthetic peptide
FAFTIPAI

SEQ ID NO: 11 - protein, 9 aa, Artificial Sequence
  synthetic peptide
GIPYNSQGQ

SEQ ID NO: 12 - protein, 10 aa, Artificial Sequence
  synthetic peptide
FEGLVDTGAD

SEQ ID NO: 13 - protein, 9 aa, Artificial Sequence
  synthetic peptide
FLQFKTWWI

SEQ ID NO: 14 - protein, 9 aa, Artificial Sequence
  synthetic peptide
VPLTKEQVR

SEQ ID NO: 15 - protein, 13 aa, Artificial Sequence
  synthetic peptide
LDLLTAEKGGLCI

SEQ ID NO: 16 - protein, 9 aa, Artificial Sequence
  synthetic peptide
TLEPIPPGE

SEQ ID NO: 17 - protein, 9 aa, Artificial Sequence
  synthetic peptide
DPLAPLQLL

SEQ ID NO: 18 - protein, 9 aa, Artificial Sequence
  synthetic peptide
KLLGDINWI

SEQ ID NO: 19 - protein, 9 aa, Artificial Sequence
  synthetic peptide
LPHSTVKTF

SEQ ID NO: 20 - protein, 9 aa, Artificial Sequence
  synthetic peptide
GPGYCSKAF

SEQ ID NO: 21 - protein, 9 aa, Artificial Sequence
  synthetic peptide
IPTRHLKFY

SEQ ID NO: 22 - protein, 9 aa, Artificial Sequence
  synthetic peptide
VPSFGRLSY

SEQ ID NO: 23 - protein, 9 aa, Artificial Sequence
  synthetic peptide
PPTVEARYK

SEQ ID NO: 24 - protein, 9 aa, Artificial Sequence
  synthetic peptide
PPESQYGYP

SEQ ID NO: 25 - protein, 9 aa, Artificial Sequence
  synthetic peptide
YPQPPTRRL

SEQ ID NO: 26 - protein, 87 aa, H. sapiens
  synthetic peptide
IIIDLKDCFFTIPLAEQDCEKFAFTIPAINNKEPATRFQWKVLPQGMLNSPTICQTFVGRALQPVREKFSDCYIIHCIDDILCAAET

SEQ ID NO: 27 - protein, 43 aa, H. sapiens
  synthetic peptide
AAIDLANAFFSIPVHKAHKKQFAFTICVYCPASGVYQQSSFVS

SEQ ID NO: 28 - protein, 58 aa, H. sapiens
FAFRWQGQQYSFTVLSQGYINSPALCHNLIQRELDHFLLLQDIILVHYIDDIMLIGSS

SEQ ID NO: 29 - protein, 71 aa, H. sapiens
KLRLPPGYFGLLLHLSQQAMKGVTVLAGVIDLDYQDEISLLLHNRGKEEYAWNTGDPLGCLLVLPCPVIKV

SEQ ID NO: 30 - protein, 35 aa, H. sapiens
YTHDRAQAVPEGTSKLHEEVAQMPMVSTPATLSLP

SEQ ID NO: 31 - protein, 179 aa, H. sapiens
SPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHCIDDILCAAETKDKLIDCYTFLQAEVANAGLAIASDKIQTSTPFHYLGMQI

SEQ ID NO: 32 - protein, 109 aa, H. sapiens
RMIVDYRKLNKGSTPTAAAVSDVVSLLEQINTSPDTWYVATDLANAFCSIPVHKAHQKQFAFGWQGQEYTFTVLSQGYINSPALCHNLVQRDLDHFSLPQDITLFHYID

SEQ ID NO: 33 - DNA, 327 bp, H. sapiens
agaatgatagtggattatcgtaagcttaacaaagggtctactccaactgcagctgctgtatcagatgtagtttcattgcttgagcaaattaacacatctcctgatacctggtatgtggccactgacttggcaaatgccttttgctccattcctgtccataaggcccaccagaagcaatttgcatttggctggcaaggccaggaatataccttcactgtcctatctcaggggtatatcaactctccagctttgtgtcataatcttgttcagagagatcttgatcacttttcacttccacaagatatcacactattccattacattgat

SEQ ID NO: 34 - protein, 584 aa, H. sapiens
MIFAGKAPSNTSTLMKFYSLLLYSLLFSFPFLCHPLPLPSYLHHTINLTHSLLAASNPSLVNNCWLCISLSSSAYTAVPAVQTDWATSPISLHLRTSFNSPHLYPPEELIYFLDRSSKTSPDISHQQAAALLRTYLKNLSPYINSTPPIFGPLTTQTTIPVAAPLCISWQRPTGIPLGNLSPSRCSFTLHLRSPTTNINETIGAFQLHITDKPSINTDKLKNISSNYCLGRHLPCISLHPWLSSPCSSDSPPRPSSCLLIPSPENNSERLLVDTRRFLIHHENRTFPSTQLPHQSPLQPLTAAALAGSLGVWVQDTPFSTPSHLFTLHLQFCLAQGLFFLCGSSTYMCLPANWTGTCTLVFLTPKIQFANGTEELPVPLMTPTQQKRVIPLIPLMVGLGLSASTVALGTGIAGISTSVMTFRSLSNDFSASITDISQTLSVLQAQVDSLAAVVLQNRRGLDLLTAEKGGLCIFLNEECCFYLNQSGLVYDNIKKLKDRAQKLANQASNYAEPPWALSNWMSWVLPIVSPLIPIFLLLLFGPCIFRLVSQFIQNRIQAITNHSIRQMFLLTSPQYHPLPQDLPSA

SEQ ID NO: 35 - DNA, 1755 bp, H. sapiens
atgatctttgctggcaaggcaccctccaatacttccaccctgatgaagttctattctttacttttatactcactcttattctcattcccattcttatgtcatcctctacctctccccagctatctccaccacactatcaaccttacccattctctcctagccgcttctaatccctccttagtgaacaactgctggctttgcatttccctttcttccagtgcctacacagctgtccccgccttacagacagactgggcaacatctcccatctccctacacctccgaacttcctttaacagccctcacctttaccctcctgaagaactcatttactttctagacaggtccagcaagacttccccagacatttcacatcagcaagctgccgccctccttcgcacttatttaaaaaacctttctccttatatcaactctactccccccatattaggacctctcacaacacaaactactattcctgtggccgctcctttgtgtatctcttggcaaagacccactggaattcccctaggtaatctttcaccttctcgatgttcctttactcttcatctccgaagtccaactacaaacatcaatgaaacaattggagccttccagctccatattacagacaagccctctatcaatactgacaaacttaaaaacattagcagtaattattgcttaggaagacacttgccctgtatttcactccatccttggctatcttccccttgctcatcagactctcctcccaggccctcttcttgtttacttatacccagccccgaaaataacagtgaaagattgctcgtagatactcgacgttttctcatacaccatgaaaatcgaaccttcccctctacgcagttaccccatcagtccccattacaacctctgacagctgccgccctagctggatccctaggagtctgggtacaagacacccctttcagcactccttctcacctttttactttacatctccagttttgcctcgcacaaggtctcttcttcctctgtggatcctctacctacatgtgcctacctgccaattggacaggcacatgtacactagtcttccttacccccaaaattcaatttgcaaatgggaccgaagagctccctgttcccctcatgacaccgacacaacaaaaaagagttattccactaattcccttgatggtcggtttaggactttctgcctccactgttgctctcggtactggaatagcaggcatttcaacgtctgtcatgaccttccgtagcctgtctaatgacttctctgctagcatcacagacatatcacaaactttatcagtcctccaggcccaagttgactctttagctgcagttgtcctccaaaaccgccgaggccttgacttactcactgctgaaaaaggaggactctgcatattcttaaatgaggagtgttgtttttacctaaatcaatctggcctggtgtatgacaacattaaaaaactcaaggatagagcccaaaaacttgccaaccaagcaagtaattacgctgaacccccttgggcactctctaattggatgtcctgggtcctcccaattgttagtcctttaatacccatttttctccttcttttatttggaccttgtatcttccgtttagtttctcaattcatccaaaaccgtatccaggccatcaccaatcattctatacgacaaatgtttcttctaacatccccacaatatcaccccttaccacaagacctcccttcagcttaa

SEQ ID NO: 36 - protein, 9 aa, Artificial Sequence
  synthetic peptide
SYFPEITHI

SEQ ID NO: 37 - protein, 87 aa, Human immunodeficiency virus
TVLDVGDAYFSVPLDEDFRKYTAFTIPSINNETPGIRYQYNVLPQGWKGSPAIFQSSMTKILEPFRKQNPDIVIYQYMDDLYVGSDL

SEQ ID NO: 38 - protein, 88 aa, Human immunodeficiency virus
MGARASVLSGGELDRWEKIRLRPGGKKKYKLKHIVWASRELERFAVNPGLLETSEGCRQILGQLQPSLQTGSEELRSLYNTVATLYCV

SEQ ID NO: 39 - protein, 10 aa, Artificial Sequence
  synthetic peptide
KEALLDTGAD

SEQ ID NO: 40 - protein, 9 aa, Artificial Sequence
  synthetic peptide
KIRLRPGGK

SEQ ID NO: 41 - protein, 9 aa, Artificial Sequence
  synthetic peptide
SSGRMIMEK

SEQ ID NO: 42 - protein, 8 aa, Human immunodeficiency virus
TAFTIPSI

SEQ ID NO: 43 - protein, 9 aa, Human immunodeficiency virus
IPLTEEAEL

SEQ ID NO: 44 - protein, 9 aa, Human immunodeficiency virus
SLYNTVATL

SEQ ID NO: 45 - protein, 9 aa, Human immunodeficiency virus
SFEPIPIHY

SEQ ID NO: 46 - protein, 9 aa, Human immunodeficiency virus
LLQLTVWGI

SEQ ID NO: 47 - protein, 9 aa, Human immunodeficiency virus
EIQKQGQGQ

SEQ ID NO: 48 - protein, 13 aa, Human immunodeficiency virus
LSHFLKEKGGLEG

SEQ ID NO: 49 - protein, 9 aa, Human immunodeficiency virus
VIYQYMDDL

SEQ ID NO: 50 - protein, 9 aa, Human immunodeficiency virus
NPDIVIYQY

SEQ ID NO: 51 - protein, 10 aa, Human immunodeficiency virus
QNIQGQMVHQ

SEQ ID NO: 52 - protein, 9 aa, Human immunodeficiency virus
TVLDVGDAY

SEQ ID NO: 53 - protein, 10 aa, Human immunodeficiency virus
VPLDEDFRKY

SEQ ID NO: 54 - protein, 10 aa, Human immunodeficiency virus
RYQYNVLPQG